(19) United States
(12) Patent Application Publication
(10) Pub. No.: US 2025/0000843 A1
(43) Pub. Date: Jan. 2, 2025

(54) LIGNAN-INSPIRED COMPOUNDS, METHODS OF MAKING, AND USING THEREOF

(71) Applicant: IOWAIBO, INC., Fairfield, IA (US)

(72) Inventor: Thomas McCorkle, Fairfield, IA (US)

(21) Appl. No.: 18/680,946

(22) Filed: May 31, 2024

Related U.S. Application Data
(60) Provisional application No. 63/524,469, filed on Jun. 30, 2023.

(51) Int. Cl. A61K 31/365; A61P 31/00

(52) U.S. Cl. CPC A61K 31/365; A61P 31/00

(57) ABSTRACT

Lignan-inspired compounds having a cancer inhibiting effect are provided. Also provided are pharmaceutical compositions comprising the compounds. Methods related to treating cancer in a subject comprising administering the compound and/or composition are also provided.

### CROSS REFERENCE TO RELATED APPLICATIONS

This application claims priority under 35 U.S.C. § 119 to provisional patent application U.S. Ser. No. 63/524,469, filed Jun. 30, 2023. The provisional patent application U.S. Ser. No. 63/524,469 is herein incorporated by reference in its entirety, including without limitation, the specification, claims, and abstract, as well as any figures, tables, appendices, or drawings thereof.

### TECHNICAL FIELD

The present disclosure relates generally to a novel lignan-inspired compound, having a dibenzyl butyl lactone motif, methods of making the novel lignan-inspired compound, and uses thereof, specifically using the novel lignan-inspired compound for inhibiting and treating breast cancer and prostate cancer cells.

### BACKGROUND

Enterolactone, and other lignans, are known to possess many valuable therapeutical properties. Urinary excretion and serum concentrations of enterolactone are low in women diagnosed with breast cancer suggesting that this lignan is chemopreventive. The inhibition of aromatase by enterolactone would suggest a mechanism by which consumption of lignan-rich plant food might contribute to reduction of estrogen-dependent diseases, such as breast cancer. The potential antioxidant activity of enterolactone could also represent a mechanism associated with the preventive action of this lignan in the development of cancers.

Enterolactone and enterolactone derivatives are promising treatments for various cancers such as breast cancer and prostate cancer. Yet, if enterolactone must be synthesized which can take many steps and costly reagents. It was surprisingly found that novel lignan-inspired compounds having a dibenzyl butyl lactone motif, have a similar chemical structure to enterolactone and provide superior treatment for breast and prostate cancer than enterolactone.

It is therefore an object of this disclosure to provide a compound which is able to produce similar cancer treating effects of enterolactone and its derivatives. It is a further objective of the disclosure to develop methods of making the novel compound. It is a further objective of the disclosure to use the novel compound to treat cancers.

Other objects, embodiments, and advantages of this disclosure will be apparent to one skilled in the art in view of the following disclosure, the drawings, and the appended claims.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

Novel pharmaceutical lignan-inspired compounds are provided herein. In certain embodiments, the pharmaceutical compound has a dibenzyl butyl lactone motif and is represented by the following formula:

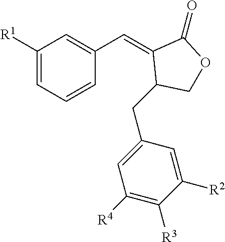

wherein R¹ and R² are independently a hydrogen, hydroxyl group or C1-22 alkyl group, and R³ and R⁴ are an ethylene oxide, dioxirane, oxetane, dioxetane, dioxolane, dihydrofuran, or furan, or its enantiomer or racemic mixture thereof, or a pharmaceutically acceptable salt thereof. In certain embodiments, R¹ is a hydroxyl group, R² is a hydrogen, and R³ and R⁴ are a dioxolane.

Pharmaceutical compositions are also provided. In certain embodiments, the composition comprises a lignan-inspired compound represented by the following formula:

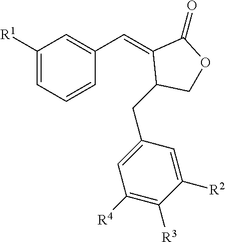

wherein R¹ and R² are independently a hydrogen, hydroxyl group or C1-22 alkyl group, and R³ and R⁴ are an ethylene oxide, dioxirane, oxetane, dioxetane, dioxolane, dihydrofuran, or furan, or its enantiomer or racemic mixture thereof, or a pharmaceutically acceptable salt thereof; and at least one pharmaceutically acceptable excipient. In certain embodiments, R¹ is a hydroxyl group, R² is a hydrogen, and R³ and R⁴ are a dioxolane.

Methods of treating cancer in a subject are also provided. In certain embodiments, the method comprises administering to the subject a composition comprising a lignan-inspired compound represented by the following formula:

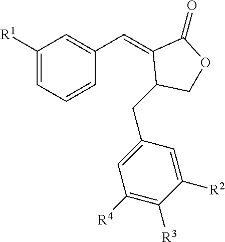

wherein R¹ and R² are independently a hydrogen, hydroxyl group or C1-22 alkyl group, and R³ and R⁴ are an ethylene oxide, dioxirane, oxetane, dioxetane, dioxolane, dihydrofuran, or furan, or its enantiomer or racemic mixture thereof, or a pharmaceutically acceptable salt thereof. In certain embodiments, R¹ is a hydroxyl group, R² is a hydrogen, and R³ and R⁴ are a dioxolane.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

FIG. 1 shows a synthesis scheme to produce an exemplary lignan-inspired novel compound.

FIG. 2 shows a synthesis scheme to produce both (R) and(S) enantiomers of an exemplary dibenzyl butyl lactone.

FIG. 3 shows a synthesis scheme to produce a racemic mixture of an exemplary dibenzyl butyl lactone.

FIGS. 4A-C show the IC50 of enterolactone in MCF7 breast cancer cells at 24 (4A), 48 (4B), and 72 (4C) hours plotted as log [inhibitor] vs. response.

FIG. 5 shows a graph of quantitative PCR-based telomeric repeat amplification protocol assay (Q-TRAP) results from in vitro treatment of MCF7 breast cancer cells with enantiomer A. Results are reported as relative telomerase activity (RTA).

FIGS. 6A-C show the IC50 of enantiomer A in MCF7 breast cancer cells at 24 (6A), 48 (6B), and 72 (6C) hours plotted as log [inhibitor] vs. response.

FIG. 7 shows a graph of Q-TRAP results from in vitro treatment of MCF7 breast cancer cells with enantiomer B. Results are reported as RTA.

FIGS. 8A-C show the IC50 of enantiomer B in MCF7 breast cancer cells at 24 (8A), 48 (8B), and 72 (8C) hours plotted as log [inhibitor] vs. response.

FIG. 9 shows a graph of Q-TRAP results from in vitro treatment of PC3 prostate cancer cells with enterolactone. Results are reported as RTA.

FIGS. 10A-C show the IC50 of enterolactone in PC3 prostate cancer cells at 24 (10A), 48 (10B), and 72 (10C) hours plotted as log [inhibitor] vs. response.

FIG. 11 shows a graph of Q-TRAP results from in vitro treatment of PC3 prostate cancer cells with enantiomer A. Results are reported as RTA.

FIGS. 12A-C show the IC50 of enantiomer A in PC3 prostate cancer cells at 24 (12A), 48 (12B), and 72 (12C) hours plotted as log [inhibitor] vs. response.

FIG. 13 shows a graph of Q-TRAP results from in vitro treatment of PC3 prostate cancer cells with enantiomer B. Results are reported as RTA.

FIGS. 14A-C show the IC50 of enantiomer A in PC3 prostate cancer cells at 24 (14A), 48 (14B), and 72 (14C) hours plotted as log [inhibitor] vs. response.

FIGS. 15A-C show graphs demonstrating the o/c ratio or developmental toxicity potential (dTP) of enantiomer B, described as “M-Peak-2” (15A), enantiomer A, described as “M-Peak-1” (15B), and (+/−) enterolactone (15C).

FIG. 16 shows a graph comparing the o/c ratio or developmental toxicity potential (dTP) of enantiomers A and B (as “M-Peak-1” and “M-Peak-2”) and (+/−) enterolactone.

FIGS. 17A-B show graphs comparing the o/c ratio or developmental toxicity potential (dTP) (17A) and the viability effect or toxicity potential (TP) (17B) of 7 common anti-cancer drugs (busulfan, fluorouracil, cytarabine, doxorubicin, methotrexate, vismodegib, and hydroxyurea), enantiomers A and B (as “M-Peak-1” and “M-Peak-2”), and (+/−) enterolactone.

An artisan of ordinary skill in the art need not view; within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments without undue experimentation, but the preferred materials and methods are described herein. In describing and claiming the embodiments, the following terminology will be used in accordance with the definitions set out below.

It is further to be understood that all terminology used herein is for the purpose of describing particular embodiments only and is not intended to be limiting in any manner or scope. For example, as used in this specification and the appended claims, the singular forms “a.” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

The methods and compositions of the present disclosure may comprise, consist essentially of, or consist of the components and ingredients of the present disclosure as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

Unless defined otherwise, all technical and scientific terms used above have the same meaning as commonly understood by one of ordinary skill in the art to which embodiments of the present disclosure pertain.

The terms “disclosure” or “present disclosure” are not intended to refer to any single embodiment of the particular disclosure but encompass all possible embodiments as described in the specification and the claims.

The term “about,” as used herein, refers to variation in the numerical quantity that can occur, for example, through typical measuring techniques and equipment, with respect to any quantifiable variable, including, but not limited to, mass, volume, time, and temperature. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

In the present disclosure, an “effective amount” or “therapeutically effective amount” of a compound or of a composition of the present disclosure is that amount of such compound and/or composition that is sufficient to effect beneficial or desired results as described herein. In terms of treatment of a mammal, e.g., a human patient, an “effective amount” is an amount sufficient to treat, reduce, manage, palliate, ameliorate, or stabilize a condition, such as a non-congenital oncosis or extended quiescence of the cells of a mammal, or both, as compared to the absence of the compound or composition.

The terms “include” and “including” when used in reference to a list of materials refer to but are not limited to the materials so listed.

The terms “derivative” or “derived” of/from a compound of the present disclosure is a derivative of or otherwise synthesized compound, which retains the backbone structure of the dibenzyl butyl lactone.

The term “enantiomer” or “optical isomer” of a compound of the present disclosure refers to two, both (R) and(S), structures, or stereoisomers, of the compound which have the same connectivity but opposite, or mirror-image, structures of one another.

As used herein, “pharmaceutically acceptable” refers to compounds, materials, compositions, and/or dosage forms which are suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. A “pharmaceutically acceptable excipient” includes any and all carriers, solvents, growth media, dispersion media, coatings, adjuvants, fillers, buffers, stabilizers, lubricants, stabilizing agents, diluents, preservatives, inactivating agents, antimicrobial, antibacterial and antifungal agents, isotonic agents, adsorption delaying agents, and the like. Such ingredients include those that are safe and appropriate for use in medical or pharmaceutical applications.

The term “racemic mixture” of a compound of the present disclosure refers to a mixture of both (R) and(S) stereoisomers, or enantiomers, of the compound in a 50:50 mixture.

The term “subject” as used herein refers to any living being that would benefit from the compositions and methods described herein. For example, the subject may be an animal, including a human, avian, bovine, canine, equine, feline, hircine, lupine, murine, ovine, and porcine animal. Subjects may also be domesticated animals such as cats, dogs, rabbits, guinea pigs, ferrets, hamsters, mice, gerbils, horses, cows, goats, sheep, donkeys, pigs, and the like. In certain embodiments, the subject is a human.

The methods and compositions may comprise, consist essentially of, or consist of the components and ingredients as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions.

### Novel Lignan-Inspired Compounds

Novel lignan-inspired compounds of the present disclosure have a dibenzyl butyl lactone structural motif. In certain embodiments, the novel lignan-inspired compound has the general structure:

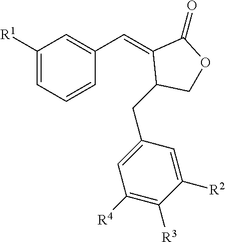

wherein R¹, R², R³, and R⁴ are independently selected from the group consisting of hydrogen, substituted or unsubstituted alkyl, substituted or unsubstituted alkenyl, substituted or unsubstituted alkynyl, substituted or unsubstituted aryl, substituted or unsubstituted heterocyclyl, substituted or unsubstituted acyl, ORa, SRa, SORa, SO₂Ra, OSO₂Ra, OSO₃Ra, NO₂, NHRa, N(Ra)₂, ═N—Ra, N(Ra)CORa, N(CORa)₂, N(Ra)SO₂R′, N(Ra)C(═NRa)N(Ra)Ra, CN, halogen, CORa, COORa, OCORa, OCOORa, OCONHRa, OCON(Ra)₂, CONHRa, CON(Ra)₂, CON(Ra)ORa, CON(Ra)SO₂Ra, PO(ORa)₂, PO(ORa)Ra, PO(ORa)(N(Ra)Ra) and aminoacid ester; and further wherein each of the Ra groups is independently selected from the group consisting of hydrogen, substituted or unsubstituted alkyl, substituted or unsubstituted alkenyl, substituted or unsubstituted alkynyl, substituted or unsubstituted aryl, and substituted or unsubstituted heterocyclyl, substituted or unsubstituted acyl, and the like; and further wherein each of the substituted or unsubstituted alkyl, alkenyl, alkynyl, aryl, heterocyclyl, and/or acyl groups are C1-22 (including all ranges therein), Moreover, when any one of R², R³, or R⁴ are cyclic groups, or wherein 2 of either R², R³, and/or R⁴ positions (i.e., R² and R³ and/or R³ and R⁴) are utilized to connect a cyclic group to the cyclohexane of the novel compound structure, both positions will be said to be the cyclic group.

“Alkyl” refers to a straight or branched hydrocarbon chain radical consisting of carbon and hydrogen atoms, containing no unsaturation, and which is attached to the rest of the molecule by a single bond, Alkyl groups may include straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (e.g., isopropyl, tert-butyl, sec-butyl, isobutyl, etc.), and alkyl-substituted alkyl groups (e.g., alkyl-substituted cycloalkyl groups and cycloalkyl-substituted alkyl groups). Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone, Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dioxolane, dihydrofuran, and furan.

Alkyl groups preferably have from 1 to about 22 carbon atoms. Methyl, ethyl, n-propyl, iso-propyl and butyl, including n-butyl, tert-butyl, sec-butyl and iso-butyl are particularly preferred alkyl groups. As used herein, the term alkyl, unless otherwise stated, refers to both cyclic and noncyclic groups, although cyclic groups will comprise at least three carbon ring members, such as cyclopropyl or cyclohexyl. Alkyl radicals may be optionally substituted by one or more substituents, such as an aryl group, like in benzyl or phenethyl.

“Alkenyl” and “Alkynyl” refer to a straight or branched hydrocarbon chain radical consisting of carbon and hydrogen atoms, containing at least one unsaturation (one carbon-carbon double or triple bond respectively) and which is attached to the rest of the molecule by a single bond. Alkenyl and alkynyl groups preferably have from 2 to about 22 carbon atoms. The terms alkenyl and alkynyl as used herein refer to both cyclic and noncyclic groups, although cyclic groups will comprise at least three carbon ring members. Alkenyl and alkenyl radicals may be optionally substituted by one or more substituents.

“Aryl” refers to a radical derived from an aromatic hydrocarbon by removal of a hydrogen atom from a ring carbon atom. Suitable aryl groups in the present disclosure include single and multiple ring compounds, including multiple ring compounds that contain separate and/or fused aryl groups. Typical aryl groups contain from 1 to 3 separated and/or fused rings and from 6 to about 22 carbon ring atoms. Aryl radicals may be optionally substituted by one or more substituents. Specially preferred aryl groups include substituted or unsubstituted phenyl, substituted or unsubstituted naphthyl, substituted or unsubstituted biphenyl, substituted or unsubstituted phenanthryl and substituted or unsubstituted anthryl.

“Heterocyclyl” refers to a cyclic radical having as ring members atoms of at least two different elements. Suitable heterocyclyl radicals include heteroaromatic and heteroalicyclic groups containing from 1 to 3 separated and/or fused rings and from 5 to about 18 ring atoms. Preferably heteroaromatic and heteroalicyclic groups contain from 5 to about 10 ring atoms. Heterocycles are described in: Katritzky, Alan R., Rees, C. W., and Scriven, E. Comprehensive Heterocyclic Chemistry (1996) Pergamon Press: Paquette, Leo A.: Principles of Modern Heterocyclic Chemistry W. A. Benjamin, New York, (1968), particularly Chapters 1, 3, 4, 6, 7, and 9): “The Chemistry of Heterocyclic Compounds. A series of Monographs” (John Wiley & Sons, New York, 1950 to present), in particular Volumes 13, 14, 16, 19, and 28. Suitable heteroaromatic groups in the compounds of the present disclosure contain one, two or three heteroatoms selected from N, O or S atoms and include, e.g., coumarinyl including 8-coumarinyl, quinolyl including 8-quinolyl, isoquinolyl, pyridyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothienyl, benzothiazolyl, benzoxazolyl, quinazolinyl, quinoxalinyl, naphthyridinyl, and furopyridinyl. Suitable heteroalicyclic groups in the compounds of the present disclosure contain one, two or three heteroatoms selected from N, O or S atoms and include, e.g., pyrrolidinyl, tetrahydrofuryl, dihydrofuryl, tetrahydrothienyl, tetrahydrothiopyranyl, piperidyl, morpholinyl, thiomorpholinyl, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, pyrazolidinyl, imidazolinyl, imidazolidinyl, 3-azabicyclo[3.1.0]hexyl, 3-azabicyclo[4.1.0]heptyl, 3H-indolyl, and quinolizinyl. Heterocyclic radicals may be optionally substituted by one or more substituents.

The novel lignan-inspired compound may be either the (R) or(S) enantiomer or a mixture of both (R) and(S) enantiomer. The mixture of enantiomers may be 90:10, 80:20, 70:30, 60:40, 40:60, 30:70, 20:80, or 10:90 of (R) and(S) enantiomers. In embodiments, the novel compound may be a racemic mixture of (R) and(S) enantiomers.

In an embodiment, the novel lignan-inspired compound has the general structure and dibenzyl butyl lactone motif as described above wherein R¹ and R² are independently a hydrogen, hydroxyl group or C1-22 alkyl group, and R³ and R⁴ are an ethylene oxide, dioxirane, oxetane, dioxetane, dioxolane, dihydrofuran, or furan. In a further embodiment, the novel lignan-inspired compound has the general structure and dibenzyl butyl lactone motif as described above wherein R¹ is a hydroxyl group or C1-22 alkyl group, R² is a hydrogen, and R³ and R⁴ are an ethylene oxide, dioxirane, oxetane, dioxetane, dioxolane, dihydrofuran, or furan.

In a preferred embodiment, the novel lignan-inspired compound has the general structure and dibenzyl butyl lactone motif as described above wherein R¹ is a hydroxyl group, R² is a hydrogen, and R³ and R⁴ are a dioxolane, which is 3-[(3,4-dimethoxyphenyl)cyclopentyl], 4-[(3-hydroxyphenol)methyl]oxolan-2-one (also described as the dibenzyl butyl lactone derivative compound). The dibenzyl butyl lactone derivative compound has the general structure:

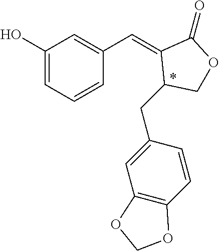 [SMILES: O=C1OCC(Cc2ccc3c(c2)OCO3)/C1=C\c1cccc(O)c1]

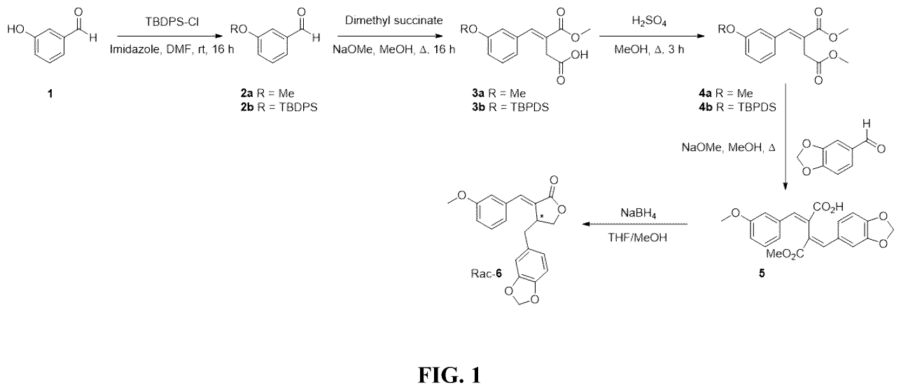

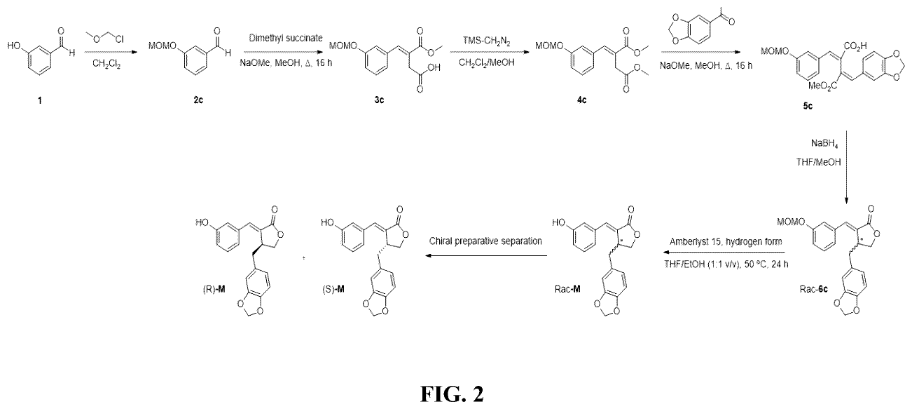

The dibenzyl butyl lactone derivative compound may be derived from natural sources or prepared by chemical synthesis. Exemplary synthesis procedures for the dibenzyl butyl lactone derivative compound may include, for example, scheme 1 as shown in FIG. 1. Scheme 1 produces a racemic mixture of a precursor of the dibenzyl butyl lactone derivative compound (described as Rac-6) via 5 steps as described by the syntheses presented in FIG. 1.

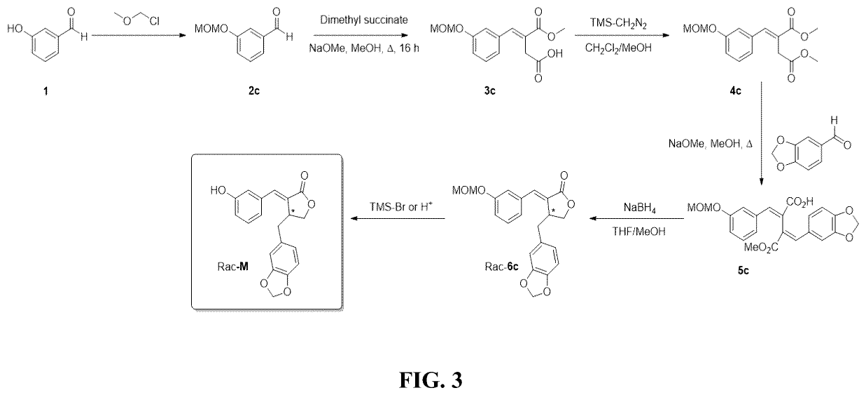

Exemplary synthesis procedures for the dibenzyl butyl lactone derivative compound may further include, for example, scheme 2 as shown in FIG. 2. Scheme 2 produces enantiomers (R and S configurations of the dibenzyl butyl lactone derivative compound, described as (R)-M and(S)-M) of 3-[(3,4-dimethoxyphenyl)cyclopentyl], 4-[(3-hydroxyphenol)methyl]oxolan-2-one (shown as Rac-M) via 7 steps as described by the syntheses presented in FIG. 2.

Exemplary synthesis procedures for the dibenzyl butyl lactone compound may further include, for example, scheme 3 as shown in FIG. 3. Scheme 3 produces a racemic mixture of the 3-[(3,4-dimethoxyphenyl)cyclopentyl], 4-[(3-hydroxyphenol)methyl]oxolan-2-one (described as Rac-M) via 6 steps as described by the syntheses presented in FIG. 3.

The dibenzyl butyl lactone derivative compound may be provided as an isolated enantiomer or as a racemic mixture thereof.

In certain embodiments, the compound is present as a pharmaceutically acceptable salt. As used herein, “pharmaceutically acceptable salts” include, for example base addition salts and acid addition salts. Pharmaceutically acceptable base addition salts may be formed with metals or amines, such as alkali and alkaline earth metals or organic amines. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N′-dibenzylethylenediamine, chloroprocaine, choline, diethanolamine, dicyclohexylamine, ethylenediamine, N-methylglucamine, and procaine.

Pharmaceutically acceptable acid addition salts include inorganic or organic acid salts. Examples of suitable acid salts include the hydrochlorides, formates, acetates, citrates, salicylates, nitrates, phosphates. Other suitable pharmaceutically acceptable salts are well known to those skilled in the art and include, for example, formic, acetic, citric, oxalic, tartaric, or mandelic acids, hydrochloric acid, hydrobromic acid, sulfuric acid or phosphoric acid: with organic carboxylic, sulfonic, sulfo or phospho acids or N-substituted sulfamic acids, for example acetic acid, trifluoroacetic acid (TFA), propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxy benzoic acid, embonic acid, nicotinic acid or isonicotinic acid; and with amino acids, such as the 20 alpha amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane 1,2-disulfonic acid, benzenesulfonic acid, 4-methylbenzenesulfonic acid, naphthalene 2-sulfonic acid, naphthalene 1,5-disulfonic acid, 2- or 3-phosphoglycerate, glucose 6-phosphate, N-cyclohexylsulfamic acid (with the formation of cyclamates), or with other acid organic compounds, such as ascorbic acid

### Pharmaceutical Compositions

Pharmaceutical compositions comprising the novel lignan-inspired compound of the present disclosure are provided. In certain embodiments, the composition comprises a compound represented by the structure of formula I:

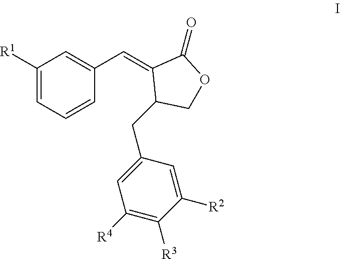

wherein R¹ and R² are independently a hydrogen, hydroxyl group or C1-22 alkyl group, and R³ and R⁴ are an ethylene oxide, dioxirane, oxetane, dioxetane, dioxolane, dihydrofuran, or furan, or its enantiomer or racemic mixture thereof, or a pharmaceutically acceptable salt thereof. In certain embodiments, R¹ is a hydroxyl group, R² is a hydrogen, and R³ and R⁴ are a dioxolane. In certain embodiments, the compound has the structure:

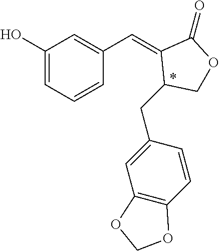 [SMILES: O=C1OCC(Cc2ccc3c(c2)OCO3)/C1=C\c1cccc(O)c1]

or its enantiomer or racemic mixture thereof, or a pharmaceutically acceptable salt thereof.

In certain embodiments, the composition comprises at least one pharmaceutically acceptable excipient. In certain embodiments, the pharmaceutically acceptable excipient may be a carrier, adjuvant, diluent, buffer, stabilizer, preservative, lubricant, and/or the like. The pharmaceutically acceptable excipient(s) may be selected based on a number of factors, including intended bioavailability of the composition, the disease, disorder or condition being treated with the composition, the species of subject, the age, size and general condition of the subject, and the route of administration. Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA): Remington's Pharmaceutical Sciences, 20th edition, pub. Lippincott, Williams & Wilkins, 2000 or Handbook of Pharmaceutical Excipients, 2nd edition, 1994.

Supplementary active ingredients also can be incorporated into the compositions. In certain embodiments, the formulation may further comprise additional ingredients, such as, for example, corn syrup solids, high-oleic safflower oil, coconut oil, soy oil, L-leucine, calcium phosphate tribasic, L-tyrosine, L-proline, L-lysine acetate, DATEM (an emulsifier), L-glutamine, L-valine, potassium phosphate dibasic, L-isoleucine, L-arginine, L-alanine, glycine, L-asparagine monohydrate, L-serine, potassium citrate, L-threonine, sodium citrate, magnesium chloride, L-histidine, L-methionine, ascorbic acid, calcium carbonate, L-glutamic acid, L-cysteine dihydrochloride, L-tryptophan, L-aspartic acid, choline chloride, taurine, m-inositol, ferrous sulfate, ascorbyl palmitate, zinc sulfate, L-carnitine, alpha-tocopheryl acetate, sodium chloride, niacinamide, mixed tocopherols, calcium pantothenate, cupric sulfate, thiamine chloride hydrochloride, vitamin A palmitate, manganese sulfate, riboflavin, pyridoxine hydrochloride, folic acid, beta-carotene, potassium iodide, phylloquinone, biotin, sodium selenate, chromium chloride, sodium molybdate, vitamin D3 and/or cyanocobalamin.

Compositions of the present disclosure may be formulated according to methods known in the art. Proper formulation is dependent upon the route of administration chosen. Suitable routes of administration include, but are not limited to, oral, parenteral (e.g., intravenous, intraarterial, rectal, subcutaneous, intramuscular, intraorbital, intracapsular, intraspinal, intraperitoneal, or intrasternal), topical (transdermal, intranasal, ocular, buccal, and sublingual), intravesical, intrathecal, enteral, pulmonary, intralymphatic, intracavital, vaginal, transurethral, intradermal, aural, intramammary, orthotopic, intratracheal, intralesional, percutaneous, endoscopical, transmucosal, and intestinal administration.

The formulations may conveniently be presented in unit dosage form. Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichloro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate. Formulations suitable for vaginal administration may be presented as pessaries, tampons, creams, gels, pastes, foams or spray formulations containing in addition to the active compound, such carriers as are known in the art to be appropriate.

The novel compounds of the present disclosure may also be preferably formulated for parenteral administration, e.g., formulated for injection via intravenous, intraarterial, subcutaneous, rectal, subcutaneous, intramuscular, intraorbital, intracapsular, intraspinal, intraperitoneal, or intrasternal routes. The compounds of the disclosure for parenteral administration comprise an effective amount of the novel compounds in a pharmaceutically acceptable carrier. Dosage forms suitable for parenteral administration include solutions, suspensions, dispersions, emulsions or any other dosage form which can be administered parenterally. Techniques and compositions for making parenteral dosage forms are known in the art.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

### Methods of Cancer Treatment

The novel lignan-inspired compounds of the present disclosure may be used in methods for the treatment of subjects having cancer. In certain embodiments the method comprises administering to the subject a composition comprising a compound represented by the structure of formula I:

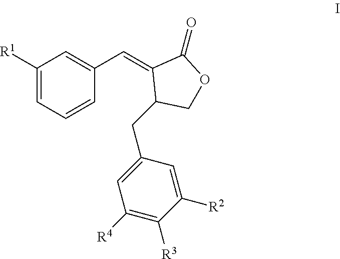

wherein R¹ and R² are independently a hydrogen, hydroxyl group or C1-22 alkyl group, and R³ and R⁴ are an ethylene oxide, dioxirane, oxetane, dioxetane, dioxolane, dihydrofuran, or furan, or its enantiomer or racemic mixture thereof, or a pharmaceutically acceptable salt thereof. In certain embodiments, R¹ is a hydroxyl group, R² is a hydrogen, and R³ and R⁴ are a dioxolane. In certain embodiments, the compound has the structure:

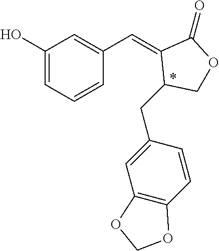 [SMILES: O=C1OCC(Cc2ccc3c(c2)OCO3)/C1=C\c1cccc(O)c1]

or its enantiomer or racemic mixture thereof, or a pharmaceutically acceptable salt thereof.

In certain embodiments, the cancer is breast cancer, prostate cancer, endometrial cancer, colorectal cancer, gastric cancer, lung cancer, liver cancer, ovarian cancer, choriocarcinoma, osteosarcoma, leukemia, and/or uterine cancer. In certain embodiments, the cancer is breast cancer or prostate cancer. In certain embodiments the cancer is metastatic cancer that has spread to other parts of the body.

Suitable routes of administration include, but are not limited to, oral, parenteral (e.g., intravenous, intraarterial, rectal, subcutaneous, intramuscular, intraorbital, intracapsular, intraspinal, intraperitoneal, or intrasternal), topical (transdermal, intranasal, ocular, buccal, and sublingual), intravesical, intrathecal, enteral, pulmonary, intralymphatic, intracavital, vaginal, transurethral, intradermal, aural, intramammary, orthotopic, intratracheal, intralesional, percutaneous, endoscopical, transmucosal, and intestinal administration.

In certain embodiments, the composition is administered orally, intravenously, or intramuscularly.

The size of the dose of each therapy which is required for the therapeutic or prophylactic treatment of a particular disease state will necessarily be varied depending on the host treated, the route of administration and the severity of the illness being treated. Accordingly, the optimum dosage may be determined by the practitioner who is treating any particular patient and taking into consideration various factors known to modify the action of drugs including severity and type of disease, body weight, sex, diet, time and route of administration, other medications and other relevant clinical factors. It may also be necessary or desirable to reduce the doses of the components of the combination treatments in order to reduce toxicity. Therapeutically effective dosages may be determined by either in vitro or in vivo methods.

In certain embodiments, an effective dose ranges from about 1 mg to about 50 mg per square meter body area of the subject. In certain embodiments, the dose ranges from about 0.05 mg/kg to about 100 mg/kg, from about 0.1 mg/kg to about 50 mg/kg, from about 0.1 mg/kg to about 25 mg/kg, from about 0.1 mg/kg to about 10 mg/kg, from about 0.1 mg/kg to about 5 mg/kg, from about 0.1 mg/kg to about 2.5 mg/kg, from about 0.1 mg/kg to about 1.5 mg/kg, from about 0.5 mg/kg to about 1.5 mg/kg, or any range therein. A unit dosage form such as a tablet, capsule, or bolus may contain, for example, about 1 mg to about 5 g of active ingredient depending on the subject.

The dosages and schedules may vary according to the particular disease state and the overall condition of the patient. Dosages and schedules may also vary if, in addition to administration of a composition of the present disclosure, one or more additional chemotherapeutic agents is/are used. Scheduling can be determined by the practitioner who is treating any particular patient.

In an embodiment, the treatment may be performed by administration of the novel compounds to the subject in need thereof or by administration directly to the site of the tumor or other cancer cells. The treatment may be performed in conjunction with administration of a chemotherapeutic agent or additional treatment agent (e.g., as part of a treatment regimen), either simultaneously, in a single composition or in separate compositions, or sequentially, with the novel compounds of the present disclosure. The treatment may be performed by administration of components in any order and in any combination. The treatment may also be performed using more than one chemotherapeutic agent, or other type of treatment. Further, the treatment may be performed by providing multiple administrations of the compositions. One skilled in the art will ascertain these variations in treatment regimens employing the novel compounds disclosed herein.

The compounds or pharmaceutical compositions of the disclosure can be used in combination with at least one or more chemotherapeutic agents. In some embodiments, the chemotherapeutic may comprise mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens, Non-limiting examples are chemotherapeutic agents, cytotoxic agents, and non-peptide small molecules such as Gleevec® (Imatinib Mesylate), Kyprolis R; (carfilzomib), Velcade® (bortezomib), Casodex (bicalutamide), Iressa® (gefitinib), Venclexta™ (venetoclax) and Adriamycin™, (docorubicin) as well as a host of chemotherapeutic agents. Non-limiting examples of chemotherapeutic agents include alkylating agents such as thiotepa and cyclosphosphamide (Cytoxan™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, chlorocyclophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; retinoic acid; esperamicins; capecitabine; and pharmaceutically acceptable salts, acids or derivatives of any of the above.

Also included as suitable chemotherapeutic cell conditioners are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO).

In certain embodiments, compounds and compositions of the present disclosure may be administered in combination with radiation therapy. Techniques for administering radiation therapy are known in the art, and these techniques can be used in the method described herein. Radiation therapy may be administered before, during, and/or after treatment with compounds and compositions of the present disclosure.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### Numbered Embodiments

The following numbered embodiments also form part of the present disclosure.

1. A pharmaceutical compound represented by the structure of formula I:

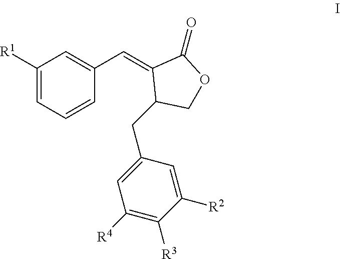

wherein:
R¹ and R² are independently a hydrogen, hydroxyl group or C1-22 alkyl group; and
R³ and R⁴ are an ethylene oxide, dioxirane, oxetane, dioxetane, dioxolane, dihydrofuran, or furan,
or its enantiomer or racemic mixture thereof, or a pharmaceutically acceptable salt thereof.

2. The compound of embodiment 1, wherein R¹ is a hydroxyl group or C1-22 alkyl group, R² is a hydrogen, and R³ and R⁴ are an ethylene oxide, dioxirane, oxetane, dioxetane, dioxolane, dihydrofuran, or furan.

3. The compound of embodiment 1, wherein R¹ is a hydroxyl group, R² is a hydrogen, and R³ and R⁴ are an ethylene oxide, dioxirane, oxetane, dioxetane, dioxolane, dihydrofuran, or furan.

4. The compound of embodiment 1, wherein R¹ is a hydroxyl group, R² is a hydrogen, and R³ and R⁴ form a dioxolane.

5. A pharmaceutical composition comprising:
a compound represented by the structure of formula I:

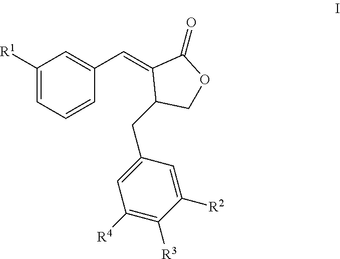

wherein:
R¹ and R² are independently a hydrogen, hydroxyl group or C1-22 alkyl group; and
R³ and R⁴ are an ethylene oxide, dioxirane, oxetane, dioxetane, dioxolane, dihydrofuran, or furan,
or its enantiomer or racemic mixture thereof, or a pharmaceutically acceptable salt thereof, or a pharmaceutically acceptable salt thereof; and
at least one pharmaceutically acceptable excipient.

6. The composition of embodiment 5, wherein R¹ is a hydroxyl group or C1-22 alkyl group, R² is a hydrogen, and R³ and R⁴ are an ethylene oxide, dioxirane, oxetane, dioxetane, dioxolane, dihydrofuran, or furan.

7. The compound of embodiment 5, wherein R¹ is a hydroxyl group, R² is a hydrogen, and R³ and R⁴ are an ethylene oxide, dioxirane, oxetane, dioxetane, dioxolane, dihydrofuran, or furan.

8. The composition of embodiment 5, wherein R¹ is a hydroxyl group, R² is a hydrogen, and R³ and R⁴ form a dioxolane.

9. The composition of any one of embodiments 5-8, wherein the pharmaceutically acceptable excipient comprises a carrier, adjuvant, diluent, buffer, stabilizer, preservative, and/or lubricant.

10. The composition of any one of embodiments 5-9, wherein the composition is formulated for oral, intravenous, or intramuscular use.

11. A method of treating cancer in a subject, comprising administering to the subject a composition comprising a compound represented by the structure of formula I:

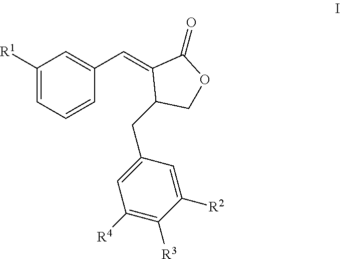

wherein:
R¹ and R² are independently a hydrogen, hydroxyl group or C1-22 alkyl group; and
R³ and R⁴ are an ethylene oxide, dioxirane, oxetane, dioxetane, dioxolane, dihydrofuran, or furan,
or its enantiomer or racemic mixture thereof, or a pharmaceutically acceptable salt thereof.

12. The method of embodiment 11, wherein R¹ is a hydroxyl group or C1-22 alkyl group, R² is a hydrogen, and R³ and R⁴ are an ethylene oxide, dioxirane, oxetane, dioxetane, dioxolane, dihydrofuran, or furan. 13. The method of embodiment 11, wherein R¹ is a hydroxyl group, R² is a hydrogen, and R³ and R⁴ form a dioxolane.

14. The method of any one of embodiments 11-13, wherein the cancer is breast cancer or prostate cancer.

15. The method of any one of embodiments 11-14, wherein the cancer is metastatic cancer.

16. The method of any one of embodiments 11-15, wherein the composition is administered orally, intravenously, or intramuscularly.

17. The method of any one of embodiments 11-16, wherein the composition is administered in combination with a chemotherapeutic agent.

18. The method of any one of embodiments 11-17, wherein the composition further comprises a pharmaceutically acceptable excipient.

19. The method of embodiment 18, wherein the pharmaceutically acceptable excipient comprises a carrier, adjuvant, diluent, buffer, stabilizer, preservative, and/or lubricant.

20. The method of any one of embodiments 11-19, wherein the subject is human.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1

### Synthesis of Racemic 6

3-Hydroxy benzaldehyde (compound 1) was reacted with tertbutyldiphenylsilylchloride (TBDPS-Cl) in the presence of imidazole in dimethylformamide (DMF) at room temperature for 16 hours. 36 g of a crude oil was obtained and confirmed that the formation of TBDPS protected compound 2b. Crude of compound 2b was purified by column chromatography. 17.2 g of a yellow oil was obtained giving a 58% yield of compound 2b. A Stobbe condensation of TBDPS protected compound 2b to ester 3b was unsuccessful. The reaction conditions resulted in hydrolysis of the TBDPS protection group and the main product that was isolated was 3-hydroxy benzaldehyde.

Instead, ester 3a was prepared starting from 2.0 g of the aldehyde 2a producing a 60% yield. The prepared mono-ester 3a was converted into the di-ester 4a producing a 40% yield over two steps. The reaction conditions for Stobbe condensation were applied on a larger scale with 10.0 g of aldehyde 2a, producing 63% yield of ester 3a, which was converted into the di-ester 4a (42% yield over two steps). Similar impurities were observed between both larger and smaller scale conversions of ester 3a to di-ester 4a. The Stobbe condensation with 9.4 g of di-ester 4a and piperonal successfully produced compound 5 with a 65% yield. Compound 5 (2.76 g) was reacted with sodium borohydride (NaBH₄) in tetrahydrofuran/methanol (THF/MeOH) at reflux.

Reduction of the unsaturated ester moiety of compound 5 was not straightforward. A few attempts with different equivalents (2, 4, and 15) of NaBH₄ in THE/MeOH mixture at reflux temperature were performed. In all of them no conversion of the starting material was observed.

Other reduction conditions, such as using different reducing agent and higher boiling solvent, were also tested using methyl benzoate as a model compound. Conditions such as reacting compound 5 with lithium borohydride (LiBH₄) in THE at elevated temperatures, as well as with NaBH₄ using diglyme as the solvent were analyzed to optimize the reaction conditions to convert compound 5 into the Rac-6.

The reduction on the model system and the reaction conditions found the optimal reaction conditions. 1.7 g of ester 5 was dissolved in 16 mL of THF and was then added in 6.0 equivalents of 1.0 g of NaBH₄, after which the reaction mixture was heated to reflux for 15 minutes. 16 mL of MeOH was added dropwise over a period of 2 hours using a syringe pump after which the reaction mixture was left to stir for 3 hours at reflux. After the 3 hours, the crude material was purified and a fraction containing the desired lactone Rac-6 product (still containing some impurities) was isolated.

The reaction conditions for the conversion of ester 5 to lactone Rac-6 were further optimized by increasing the time of addition of MeOH to the reaction mixture to 4 hours instead of 2 hours and by increasing the reaction time at reflux to 16 hours instead of 3 hours. Using these reaction conditions, 1.7 g ester 5 was converted into the lactone Rac-6, which was purified to produce a 11% yield of pure Rac-6. LCMs analysis of the purified Rac-6 displayed a purity of 98.8% (at 286 nm). UPC analysis confirmed the racemic nature of lactone Rac-6. Both enantiomers elute respectively at 6.00 minutes (m/z: 339.13 [M+1]) and 6.42 minutes (m/z: 339.14 [M+1]).

The final path of synthesis to produce Rac-6 from 3-Hydroxy benzaldehyde (compound 1) is shown in Scheme 1 of FIG. 1.

### Example 2

### Synthesis of the Dibenzyl Butyl Lactone Derivative Compound

Based on the difficulties in maintaining the TBDPS protection group above in Example 1, other protecting groups for the alcohol moiety in compound 1 were considered, for example methoxy methyl ether (MOM). Aldehyde 2c (96% crude yield) was prepared starting from 10.0 g of aldehyde 1. A MOM protecting group would likely be stable under basic conditions that are required for Stobbe condensation. To avoid cleavage of the MOM protecting group under acidic conditions, MOM protected ester 3c (28% yield) was prepared starting from 5.0 g of aldehyde 2c. 500 mg of ester 3c by reacting the compound with (trimethylsilyl)diazomethane in CH₂Cl₂/MeOH was successfully alkylated and the di-ester 4c (78% yield) was produced. MOM protected ester 5c (66% yield) was prepared starting from 5.0 g of di-ester 4c.

The reaction conditions used for the conversion of ester 5c to lactone Rac-6 (Scheme 1 of FIG. 1) were used in an attempt to prepare lactone Rac-6c starting from 1.9 g of ester 5c (Scheme 2 of FIG. 2). In addition, the same reaction was repeated starting from 1.2 g of ester 5c using 10 equivalents of NaBH₄. ¹H-NMR analysis of both crude reaction mixtures show the presence of lactone Rac-6c.

A new batch of MOM protected ester 5c (72% yield) was prepared starting from 4.8 g of di-ester 4c. The conversion of ester 5c to Rac-6c was studied using different reaction conditions in an attempt to optimize the yield. The conditions are summarized in Table 1.

TABLE 1

| | | | Addition time |
|---|---|---|---|
| | Ester | | of 16 mL |
| # | 5 c (g) | NaBH₄ (eq.) | MeOH (h) |
| I | 1.9 | 6.0 | 4 |
| II | 1.2 | 10.0 | 4 |
| III | 1.5 | 10.0 | 10 |

Both crude reaction mixtures obtained while using the reaction conditions described in #I and #II show the presence of lactone Rac-6c. However, the majority of both mixtures still consisted of presumably an intermediate compound. The crude mixture obtained with the reaction conditions of #II appeared cleaner and the presence of lactone Rac-6c was more significant. The unreacted intermediate compound could be removed from the crude reaction mixture obtained with the reaction conditions of #II by eluting the crude oil with CH₂Cl₂ over a SiO₂ plug. Further purification was attempted via ISCO chromatography.

The crude mixture of Rac-6c was treated with Amberlyst® 15 (1,2-bis(ethenyl)benzene: 2-ethenylbenzene sulfonic acid) (hydrogen form) in THF/MeOH for 16 hours at room temperature to remove the MOM group of Rac-6c. The crude reaction mixture was then suspended in CHCl₃ after which 29 mg of goal compound Rac-M, the dibenzyl butyl lactone derivative compound (3-[(3,4-dimethoxyphenyl)cyclopentyl], 4-[(3-hydroxyphenol)methyl]oxolan-2-one) could be isolated by filtration.

The final path of synthesis to produce Rac-M (or 3-[(3,4-dimethoxyphenyl)cyclopentyl], 4-[(3-hydroxyphenol)methyl]oxolan-2-one) from 3-Hydroxy benzaldehyde (compound 1) is shown in Scheme 2 of FIG. 2.

Similarly, the crude mixture of Rac-6c was treated with TMS-Br or H⁺ to remove the MOM group of Rac-6c. This path of synthesis to produce Rac-M (or 3-[(3,4-dimethoxyphenyl)cyclopentyl], 4-[(3-hydroxyphenol)methyl]oxolan-2-one) from 3-Hydroxybenzaldehyde (compound 1) is shown in Scheme 3 of FIG. 3.

### Example 3

### The Dibenzyl Butyl Lactone Derivative Exhibits Toxicity to Breast Cancer Cells

Toxicity of the dibenzyl butyl lactone derivative (3-[(3,4-dimethoxyphenyl)cyclopentyl], 4-[(3-hydroxyphenol)methyl]oxolan-2-one) in breast cancer cells was evaluated using MTT assay. The MTT toxicity test is a colorimetric assay to measure cells' metabolic activity by serving as a substrate of cellular enzymes that reduce the tetrazolium orange dye, MTT (3-(4,5-Dimethylthiazol-2-yl)-2,5-diphenyltetrazolium bromide) to its insoluble, formazan, giving a purple color. The rate of conversion is directly proportional to cells' mitochondrial metabolic activity via NAD (P) H-dependent cellular oxidoreductase enzymes and serves as a surrogate marker of cell viability.

### Materials and Methods

Cultures of MCF7 (human breast cancer cell line) working stock (5 passages) were established under standard culture conditions in Eagle's minimum essential medium (EMEM). EMEM was supplemented with 10% fetal bovine serum (FBS), 2 mM Glutamine and Pen/Strep, and Penicillin 0.01 mg/ml.

Cells previously expanded were seeded in 96 well plates at 1.9×10⁵ and 9.6×10⁴ cells/plate for 24- and 72-hours treatment respectively. This concentration is known to show the best window for the MTT assay, with better sensitivity and low variability.

5 mg of powdered enterolactone, dibenzyl butyl lactone derivative enantiomer A, and dibenzyl butyl lactone derivative enantiomer B were dissolved in 500 μl of DMSO at room temperature. Eight-point curves, ⅓ dilutions (highest concentration 125 μM), were prepared. Final concentration of DMSO was 0.5%, a concentration that does not affect the cells.

Twenty-four hours following the seeding process, the cells were washed once with phosphate buffered saline (PBS) and treated with the respective compounds in cell culture media. Each condition was tested in triplicates. For positive and negative controls, 8 mM methyl methane sulfonate (MMS) and DMSO 100% were used respectively.

Following compound addition, the plates were incubated for 24 and 72 hours. After the treatment period, cells were washed twice with PBS and media was replaced with MTT reagent at 0.5 mg/ml in Dulbecco's modified Eagle medium (DMEM) without phenol red. The plates were gently shaken and incubated for 4 hours. After the incubation the medium was removed and replaced by DMSO. The plates were gently shaken to solubilize the formazan crystals. Absorbance was measured using an Envision multiplate reader at a wavelength of 570 nm.

### Results

Eight serial dilutions were prepared (125.00, 41.67, 13.89, 4.63, 1.54, 0.51, 0.17 and 0.06 UM to evaluate the compounds (in triplicate). MTT results showing cell death values above 20% are considered as having a significant toxic effect.

MTT assay results are shown in Tables 2-7 and expressed as the percentage of cell death after treatment.

TABLE 2
24 hours-Enterolactone

| | Cell | Cell | Cell | | |
|---|---|---|---|---|---|
| Concentration | death #1 | death #2 | death #3 | | |
| (μM) | (%) | (%) | (%) | MEAN | SD |
| 125.00 | −13.51 | −8.13 | 7.64 | −4.66 | 10.99 |
| 41.67 | −30.05 | −30.43 | −40.43 | −33.64 | 5.89 |
| 13.89 | −10.05 | −8.13 | −3.51 | −7.23 | 3.36 |
| 4.63 | −5.05 | −19.28 | −25.82 | −16.71 | 10.62 |
| 1.54 | −16.20 | −16.97 | −21.20 | −18.13 | 2.69 |
| 0.51 | 2.26 | −23.51 | −19.28 | −13.51 | 13.82 |
| 0.17 | −14.66 | −12.36 | −5.43 | −10.82 | 4.80 |
| 0.06 | −11.97 | −41.20 | −33.13 | −28.77 | 15.10 |

TABLE 3
24 hours-Enantiomer A

| | Cell | Cell | Cell | | |
|---|---|---|---|---|---|
| Concentration | death #1 | death #2 | death #3 | | |
| (μM) | (%) | (%) | (%) | MEAN | SD |
| 125.00 | 98.41 | 95.72 | 100.72 | 98.29 | 2.50 |
| 41.67 | −83.51 | −78.89 | −45.05 | −69.15 | 21.00 |
| 13.89 | −61.20 | −35.05 | −25.05 | −40.43 | 18.67 |
| 4.63 | −58.89 | −57.74 | −34.66 | −50.43 | 13.67 |
| 1.54 | −48.89 | −51.59 | −43.51 | −48.00 | 4.11 |
| 0.51 | −57.74 | −18.51 | −45.82 | −40.69 | 20.11 |
| 0.17 | −27.36 | −44.28 | −55.05 | −42.23 | 13.96 |
| 0.06 | −25.82 | −37.36 | −35.43 | −32.87 | 6.18 |

TABLE 4
24 hours-Enantiomer B

| | Cell | Cell | Cell | | |
|---|---|---|---|---|---|
| Concentration | death #1 | death #2 | death #3 | | |
| (μM) | (%) | (%) | (%) | MEAN | SD |
| 125.00 | 63.85 | 47.46 | 63.13 | 58.15 | 9.26 |
| 41.67 | −211.84 | −192.61 | −204.01 | −202.82 | 9.67 |
| 13.89 | −132.06 | −174.09 | −134.91 | −147.02 | 23.49 |
| 4.63 | −134.19 | −163.40 | −138.47 | −145.35 | 15.77 |
| 1.54 | −115.67 | −156.99 | −114.96 | −129.21 | 24.06 |
| 0.51 | −124.22 | −120.66 | −95.01 | −113.30 | 15.93 |
| 0.17 | −146.30 | −115.67 | −91.45 | −117.81 | 27.49 |
| 0.06 | −90.03 | −85.75 | −79.34 | −85.04 | 5.38 |

TABLE 5
72 hours-Enterolactone

| | Cell | Cell | Cell | | |
|---|---|---|---|---|---|
| Concentration | death #1 | death #2 | death #3 | | |
| (μM) | (%) | (%) | (%) | MEAN | SD |
| 125.00 | 88.19 | 90.54 | 89.20 | 89.31 | 1.18 |
| 41.67 | −22.36 | −24.04 | −22.70 | −23.03 | 0.89 |
| 13.89 | −48.83 | −38.44 | −39.11 | −42.13 | 5.81 |
| 4.63 | −53.85 | −66.92 | −69.26 | −63.34 | 8.30 |
| 1.54 | −70.27 | −54.19 | −60.89 | −61.78 | 8.08 |
| 0.51 | −66.25 | −61.22 | −61.89 | −63.12 | 2.73 |
| 0.17 | −67.59 | −62.23 | −65.91 | −65.24 | 2.74 |
| 0.06 | −30.40 | −23.70 | −42.46 | −32.19 | 9.51 |

TABLE 6
72 hours-Enantiomer A

| | Cell | Cell | Cell | | |
|---|---|---|---|---|---|
| Concentration | death #1 | death #2 | death #3 | | |
| (μM) | (%) | (%) | (%) | MEAN | SD |
| 125.00 | 99.92 | 99.92 | 99.58 | 99.80 | 0.19 |
| 41.67 | 15.49 | 4.10 | 2.76 | 7.45 | 7.00 |
| 13.89 | −43.13 | −52.85 | −46.15 | −47.38 | 4.97 |
| 4.63 | −90.37 | −113.15 | −79.31 | −94.28 | 17.25 |
| 1.54 | −57.87 | −73.62 | −58.88 | −63.46 | 8.81 |
| 0.51 | −79.98 | −51.84 | −72.61 | −68.15 | 14.59 |
| 0.17 | −56.87 | −58.21 | −50.17 | −55.08 | 4.31 |
| 0.06 | −51.17 | −29.40 | −11.64 | −30.74 | 19.80 |

TABLE 7
72 hours-Enantiomer B

| | Cell | Cell | Cell | | |
|---|---|---|---|---|---|
| Concentration | death #1 | death #2 | death #3 | | |
| (μM) | (%) | (%) | (%) | MEAN | SD |
| 125.00 | 99.00 | 99.52 | 99.00 | 99.17 | 0.30 |
| 41.67 | −18.96 | −6.08 | 17.10 | −2.65 | 18.27 |
| 13.89 | −23.34 | −30.55 | −9.69 | −21.20 | 10.60 |
| 4.63 | −32.87 | −34.42 | −30.04 | −32.44 | 2.22 |
| 1.54 | −22.57 | −37.77 | −19.48 | −26.60 | 9.79 |
| 0.51 | −28.49 | −52.45 | −56.31 | −45.75 | 15.07 |
| 0.17 | −74.60 | −57.08 | −52.45 | −61.38 | 11.68 |
| 0.06 | −31.84 | −27.21 | −56.05 | −38.37 | 15.49 |

Morphological analysis through optical microscopy did not show any deleterious effect at any concentration. No compound precipitation was observed at any concentration.

These results indicate that at higher concentrations, dibenzyl butyl lactone derivative enantiomers A and B exhibit increased breast cancer cell death as compared to enterolactone at both 24 hours and 72 hours post-treatment.

### Example 4

### The Dibenzyl Butyl Lactone Derivative Compound Inhibits Telomerase in Breast Cancer Cells

Telomeric repeat amplification protocol (TRAP) was modified for quantitative real-time PCR (Q-TRAP) and was used to determine the effect of the dibenzyl butyl lactone derivative (3-[(3,4-dimethoxyphenyl)cyclopentyl], 4-[(3-hydroxyphenol)methyl]oxolan-2-one) on telomerase activity in breast cancer cells. In this assay telomerase activity is determined by Q-TRAP in MCF7 after 24, 48 and 72 hours after treatment and at concentrations of 40 μM, 20 μM, 10 μM, 5 μM, and 2.5 μM. Cells were seeded at 300.000 (24 hours), 200.000 (48 hours), 150.000 (72 hours) cells/well in EMEM).

The general mechanism of the Q-TRAP technique involves cellular pellets being lysed for protein extraction which is then subsequently quantified and stored under specific conditions to avoid protein degradation. Protein obtained in the process was used within 72 hours and samples were stored at 4° C. Telomerase protein extracts are then incubated with a specific oligonucleotide substrate to allow the enzymatic addition of telomeric DNA repeats by endogenous telomerase. Following the enzymatic reaction, telomerase extension products are then amplified and quantified by real-time qPCR. In real time PCR, a positive reaction is detected by accumulation of a fluorescent signal. The Ct (cycle threshold) is defined as the number of cycles required for fluorescence to cross the threshold (i.e.: exceeds background levels). The telomerase-positive standard dilution series is plotted against the telomere protein concentration (r₂>0.9) as a standard curve of Ct values.

Assays were performed in triplicate. The mean and standard deviation (SD) from each triplicate is calculated which include both positive (Lymphoid cell line standard Curve) and negative controls (inactivated by heat). Data are reported as relative telomerase activity (RTA). For clarity, Table 8 presents abbreviations used throughout the results and figures.

TABLE 8

| Treatment | Abbreviation |
|---|---|
| Control untreated group | Ctrl |
| Enterolactone at 40 μM | O_40 |
| Enterolactone at 20 μM | O_20 |
| Enterolactone at 10 μM | O_10 |
| Enterolactone at 5 μM | O_5 |
| Enterolactone at 2.5 μM | O_2.5 |
| A enantiomer at 40 μM | A_40 |
| A enantiomer at 20 μM | A_20 |
| A enantiomer at 10 μM | A_10 |
| A enantiomer at 5 μM | A_5 |
| A enantiomer at 2.5 μM | A_2.5 |
| B enantiomer at 40 μM | B_40 |
| B enantiomer at 20 μM | B_20 |
| B enantiomer at 10 μM | B_10 |
| B enantiomer at 5 μM | B_5 |
| B enantiomer at 2.5 μM | B_2.5 |

Only those samples with sufficient amount of protein were analyzed (>0.3 mg/ml). Standard curve results were generated by graphing Threshold cycles (Ct values) of HeLa cell line standards against log of 1000, 333, 111, 37.03, 48.34, 4.11, 1.37 and 0.45 ng of protein (whole cell extract). The cycle number at the threshold (Ct value) for each sample was interpolated in the curve and used to calculate RTA.

Calculated RTA values and Q-TRAP results for enterolactone showed there was no significant difference in telomerase activity following treatment with enterolactone as compared to control (untreated) groups.

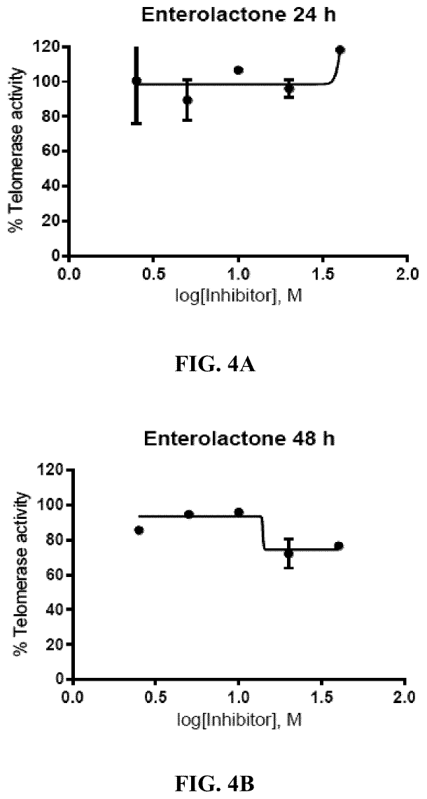

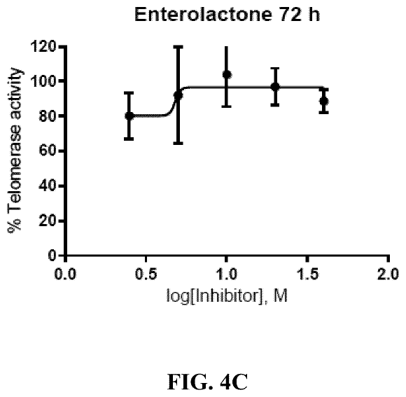

Graphs of IC50 of enterolactone at 24, 48, and 72 hours were generated using a nonlinear regression and plotted as log [inhibitor] vs. response (FIGS. 4A-C). The average of telomerase activity from replicates were calculated for each concentration of enterolactone at 24, 48 and 72h and compared to the average value of the no-drug control to generate the percent (%) of telomerase activation and plotted against the treatment concentration to determine the drug concentrations for each formulation to achieve 50% inhibition (IC50 value), as shown in Table 9.

TABLE 9
IC50 μM-Enterolactone

| | 24 h | 48 h | 72 h |
|---|---|---|---|
| Enterolactone | >40 | >40 | >40 |

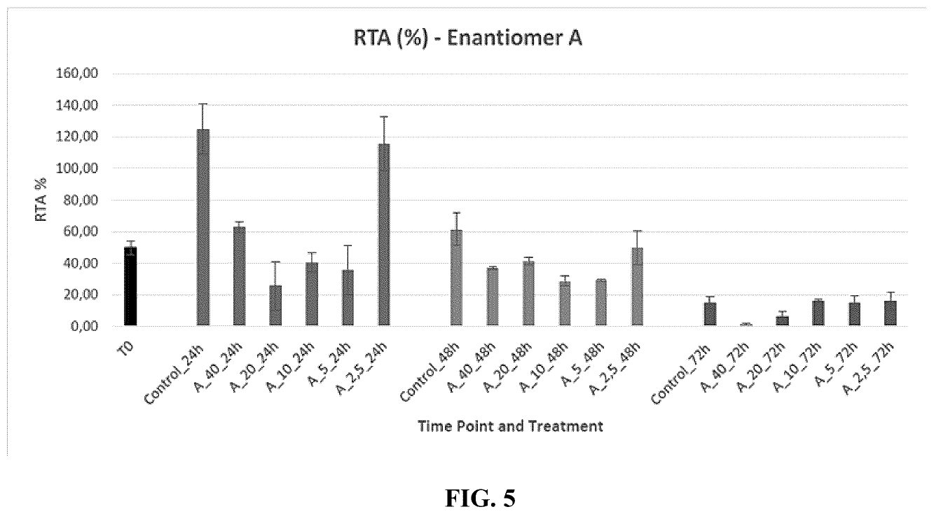

Calculated RTA values for all dibenzyl butyl lactone derivative enantiomer A treated samples are shown in Table 10. Q-TRAP results are shown in FIG. 5.

TABLE 10
Telomerase Activity (RTA %) - Enantiomer A

| | 0 Hours | | 24 Hours | | 48 Hours | | 72 Hours | |
|---|---|---|---|---|---|---|---|---|
| | RTA | | RTA | | RTA | | RTA | |
| | (%) | SD | (%) | SD | (%) | SD | (%) | SD |
| Control | 49.71 | 4.22 | 124.84 | 15.84 | 61.23 | 10.44 | 14.77 | 3.80 |
| A_40 | | | 62.53 | 3.35 | 36.82 | 0.68 | 0.86 | 0.76 |
| A_20 | | | 25.50 | 15.27 | 41.20 | 2.49 | 5.82 | 3.45 |
| A_10 | | | 40.24 | 6.05 | 28.47 | 3.16 | 15.87 | 0.72 |
| A_5 | | | 35.54 | 15.64 | 29.14 | 0.43 | 14.77 | 4.27 |
| A_2.5 | | | 115.47 | 17.18 | 49.73 | 10.70 | 15.70 | 5.76 |

Data were grouped by time point of treatment and statistical analysis for grouped data was applied using t-test comparing with a control condition. Results are shown in Table 11.

TABLE 11
T-Student analysis indicates if there are significant differences among the Q-TRAP
results (RTA) compared to control condition. Significant differences are indicated in the
“Significance” column. From lowest to highest significance: No: non-significant; Yes (*):
p < 0.05; Yes (**): p < 0.01; Yes (***): p < 0.001; Yes (****): p < 0.0001.
T-Student Analysis-Relative Telomerase Activity
Enantiomer A

| 24 hours | RTA (p Value) | Significance |
|---|---|---|
| Control vs. A_40 | 0.032 | YES (*) |
| Control vs. A_20 | 0.024 | YES (*) |
| Control vs. A_10 | 0.019 | YES (*) |
| Control vs. A_5 | 0.030 | YES (*) |
| Control vs. A_2.5 | 0.628 | NO |
| 48 hours | RTA (p Value) | Significance |
| Control vs. A_40 | 0.052 | YES (*) |
| Control vs. A_20 | 0.085 | NO |
| Control vs. A_10 | 0.007 | YES (**) |
| Control vs. A_5 | 0.026 | YES (*) |
| Control vs. A_2.5 | 0.317 | NO |
| 72 hours | RTA (p Value) | Significance |
| Control vs. A_40 | 0.003 | YES (**) |
| Control vs. A_20 | 0.076 | NO |
| Control vs. A_10 | 0.726 | NO |
| Control vs. A_5 | 0.999 | NO |
| Control vs. A_2.5 | 0.827 | NO |

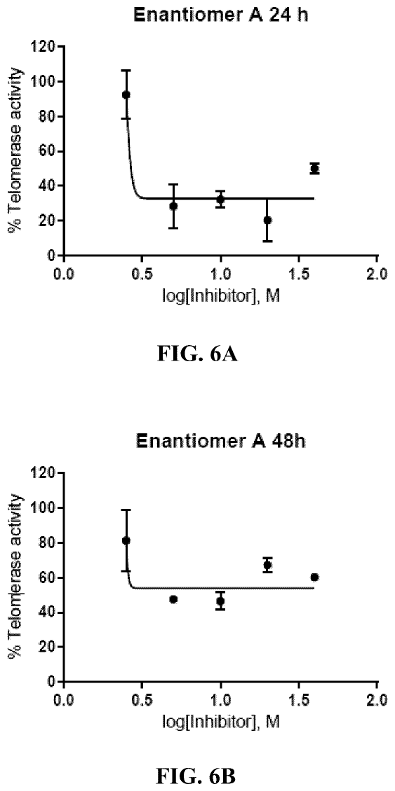

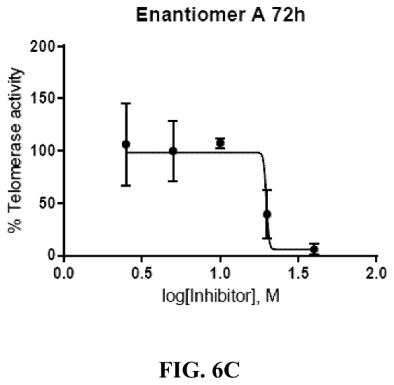

Graphs of IC50 of dibenzyl butyl lactone derivative enantiomer A at 24, 48, and 72 hours were generated using a nonlinear regression and plotted as log [inhibitor] vs. response (FIGS. 6A-C). The average of telomerase activity from replicates were calculated for each concentration of dibenzyl butyl lactone derivative enantiomer A at 24, 48 and 72h and compared to the average value of the no-drug control to generate the percent (%) of telomerase activation and plotted against the treatment concentration to determine the drug concentrations for each formulation to achieve 50% inhibition (IC50 value), as shown in Table 12.

TABLE 12
IC50 μM-Enantiomer A

| | 24 h | 48 h | 72 h |
|---|---|---|---|
| Enantiomer | ~2.49 | 2.23 | 19.79 |
| A | | | |

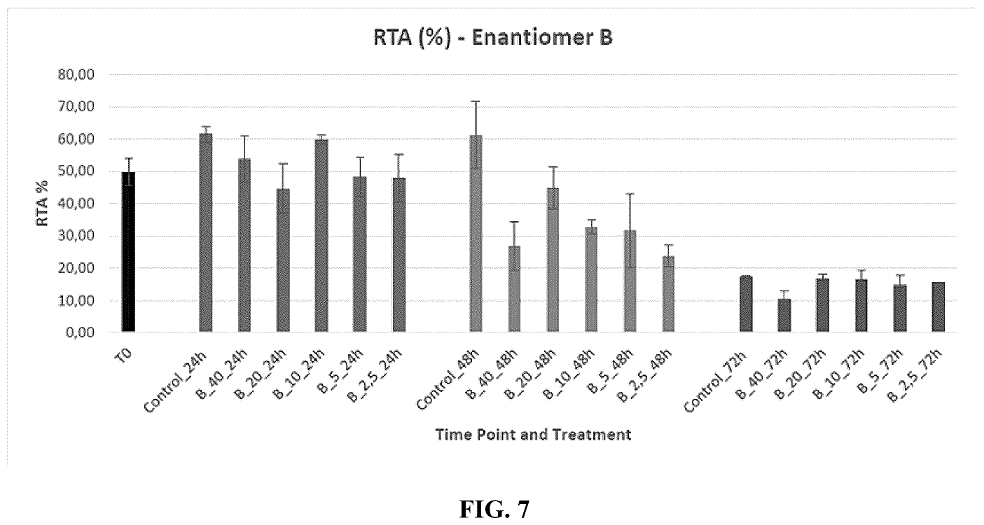

Calculated RTA values for all dibenzyl butyl lactone derivative enantiomer B treated samples are shown in Table 13. Q-TRAP results are shown in FIG. 7.

TABLE 13
Telomerase activity (RTA %) - Enantiomer B

| | 0 Hours | | 24 Hours | | 48 Hours | | 72 Hours | |
|---|---|---|---|---|---|---|---|---|
| | RTA | | RTA | | RTA | | RTA | |
| | (%) | SD | (%) | SD | (%) | SD | (%) | SD |
| Control | 49.71 | 4.22 | 61.47 | 2.53 | 61.23 | 10.44 | 17.16 | 0.49 |
| B_40 | | | 53.63 | 7.32 | 26.82 | 7.60 | 10.17 | 2.75 |
| B_20 | | | 44.48 | 7.66 | 44.82 | 6.57 | 16.69 | 1.45 |
| B_10 | | | 59.90 | 1.49 | 32.76 | 2.25 | 16.26 | 3.01 |
| B_5 | | | 48.30 | 6.02 | 31.70 | 11.45 | 14.68 | 3.13 |
| B_2.5 | | | 47.79 | 7.30 | 23.71 | 3.41 | 15.49 | 0.02 |

Data were grouped by time point of treatment and statistical analysis for grouped data was applied using t-test comparing with a control condition. Results are shown in Table 14.

TABLE 14
T-Student analysis indicates if there are significant differences among the Q-TRAP
results (RTA) compared to control condition. Significant differences are indicated in the
“Significance” column. From lowest to highest significance: No: non-significant; Yes (*):
p < 0.05; Yes (**): p < 0.01; Yes (***): p < 0.001; Yes (****): p < 0.0001.
T-Student Analysis-Relative Telomerase Activity
Enantiomer B

| 24 hours | RTA (p Value) | Significance |
|---|---|---|
| Control vs. B_40 | 0.289 | YES (*) |
| Control vs B_20 | 0.097 | YES (*) |
| Control vs. B_10 | 0.528 | YES (*) |
| Control vs. B_5 | 0.104 | YES (*) |
| Control vs. B_2.5 | 0.129 | NO |
| 48 hours | RTA (p Value) | Significance |
| Control vs. B_40 | 0.029 | YES (*) |
| Control vs B_20 | 0.150 | NO |
| Control vs. B_10 | 0.036 | YES (*) |
| Control vs. B_5 | 0.058 | NO |
| Control vs. B_2.5 | 0.018 | YES (*) |
| 72 hours | RTA (p Value) | Significance |
| Control vs. B_40 | 0.072 | NO |
| Control vs B_20 | 0.710 | NO |
| Control vs. B_10 | 0.718 | NO |
| Control vs. B_5 | 0.384 | NO |
| Control vs. B_2.5 | 0.041 | YES (*) |

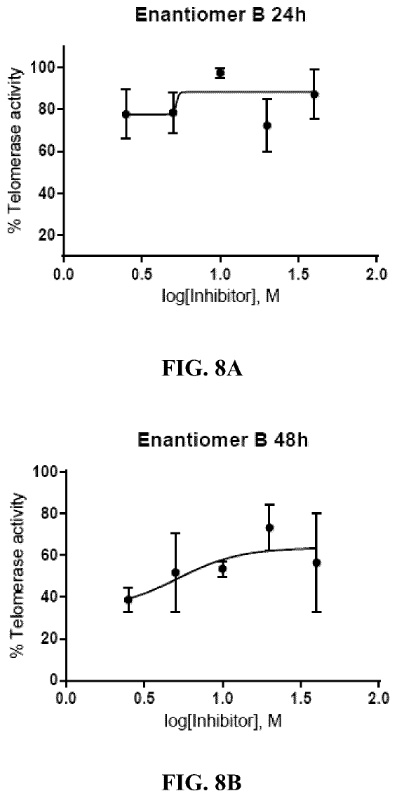

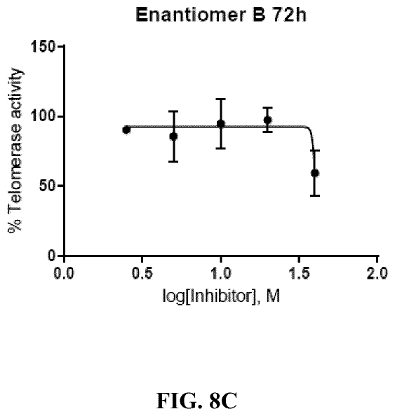

Graphs of IC50 of dibenzyl butyl lactone derivative enantiomer B at 24, 48, and 72 hours were generated using a nonlinear regression and plotted as log [inhibitor] vs. response (FIGS. 8A-C). The average of telomerase activity from replicates were calculated for each concentration of dibenzyl butyl lactone derivative enantiomer B at 24, 48 and 72h and compared to the average value of the no-drug control to generate the percent (%) of telomerase activation and plotted against the treatment concentration to determine the drug concentrations for each formulation to achieve 50% inhibition (IC50 value), as shown in Table 15.

TABLE 15
IC50 μM-Enantiomer B

| | 24 h | 48 h | 72 h |
|---|---|---|---|
| Enantiomer | >40 | 5.165 | >40 |
| B | | | |

### Discussion

At 24 hours, all groups treated with dibenzyl butyl lactone derivative enantiomer A showed lower telomerase activity levels compared to control. The differences were significant for all groups except for the lowest concentration. At 48 hours, all treated groups showed lower telomerase activity levels compared to control. The differences were significant for three of the treated groups. At 72 hours, although all RTA levels dropped to basal levels it can still be observed that the groups treated with the two highest concentrations presented further lower RTA levels with the difference between the control group and the highest concentration being statistically significant.

At 24 hours, all groups treated with dibenzyl butyl lactone derivative enantiomer B showed lower telomerase activity levels compared to control with various differences being significant. At 48 hours, all treated groups showed lower telomerase activity levels compared to control, even more pronounced than 24 hours, with various differences being significant. At 72 hours, all RTA levels dropped to basal levels. Nevertheless, the group treated with the highest concentration presented lower RTA levels.

As a whole, these results show that both dibenzyl butyl lactone derivative enantiomer A and B exhibit a telomerase inhibition effect in MCF7 breast cancer cells.

### Example 5

### The Dibenzyl Butyl Lactone Derivative Exhibits Toxicity to Prostate Cancer Cells

Toxicity of the dibenzyl butyl lactone derivative (3-[(3,4-dimethoxyphenyl)cyclopentyl], 4-[(3-hydroxyphenol)methyl]oxolan-2-one) in prostate cancer cells was evaluated using MTT assay. The same methods as those used in Example 2 were employed, except the cell line used was PC3, a human prostate cancer cell line, and cell cultures were established in Kaighn's modification of Ham's F-48 medium.

MTT assay results are shown in Tables 16-21 and expressed as the percentage of cell death after treatment.

TABLE 16
24 hours-Enterolactone

| | Cell | Cell | Cell | | |
|---|---|---|---|---|---|
| Concentration | death | death #2 | death | | |
| (μM) | #1 (%) | (%) | #3 (%) | MEAN | SD |
| 125.00 | 19.65 | 12.01 | 15.10 | 15.59 | 3.84 |
| 41.67 | −32.30 | −17.98 | −35.59 | −28.62 | 9.36 |
| 13.89 | −33.85 | −41.29 | −40.62 | −38.59 | 4.12 |
| 4.63 | −41.49 | −38.59 | −32.20 | −37.42 | 4.75 |
| 1.54 | −30.56 | −47.58 | −25.82 | −34.65 | 11.45 |
| 0.51 | −29.78 | −35.88 | −8.98 | −24.88 | 14.10 |
| 0.17 | −12.56 | −5.41 | −10.24 | −9.40 | 3.65 |
| 0.06 | −3.37 | −20.79 | −22.43 | −15.53 | 10.56 |

TABLE 17
24 hours-Enantiomer A

| | Cell | Cell | Cell | | |
|---|---|---|---|---|---|
| Concentration | death | death #2 | death | | |
| (μM) | #1 (%) | (%) | #3 (%) | MEAN | SD |
| 125.00 | 32.42 | 28.74 | 33.00 | 31.39 | 2.31 |
| 41.67 | −6.18 | −0.76 | −12.85 | −6.60 | 6.06 |
| 13.89 | −26.49 | −20.40 | −18.56 | −21.82 | 4.15 |
| 4.63 | −17.98 | −21.37 | −15.27 | −18.21 | 3.05 |
| 1.54 | −29.20 | −24.37 | −23.78 | −25.78 | 2.97 |
| 0.51 | −2.70 | −15.18 | −10.44 | −9.44 | 6.30 |
| 0.17 | −27.17 | −17.50 | −8.79 | −17.82 | 9.19 |
| 0.06 | −10.92 | −13.82 | −11.02 | −11.92 | 1.65 |

TABLE 18
24 hours-Enantiomer B

| | Cell | Cell | Cell | | |
|---|---|---|---|---|---|
| Concentration | death | death #2 | death | | |
| (μM) | #1 (%) | (%) | #3 (%) | MEAN | SD |
| 125.00 | 6.23 | 3.54 | 4.64 | 4.80 | 1.36 |
| 41.67 | −68.77 | −35.80 | −33.35 | −45.97 | 19.78 |
| 13.89 | −71.22 | −63.50 | −63.74 | −66.15 | 4.39 |
| 4.63 | −72.93 | −63.74 | −39.60 | −58.76 | 17.22 |
| 1.54 | −58.35 | −51.85 | −43.77 | −51.32 | 7.31 |
| 0.51 | −15.09 | −34.33 | −34.94 | −28.12 | 11.29 |
| 0.17 | −22.81 | −20.24 | −14.35 | −19.13 | 4.33 |
| 0.06 | −14.60 | −12.15 | −1.24 | −9.33 | 7.11 |

TABLE 19
72 hours-Enterolactone

| | Cell | Cell | Cell | | |
|---|---|---|---|---|---|
| Concentration | death | death #2 | death | | |
| (μM) | #1 (%) | (%) | #3 (%) | MEAN | SD |
| 125.00 | 35.94 | 32.46 | 22.11 | 30.17 | 7.19 |
| 41.67 | −35.45 | −32.21 | −34.95 | −34.20 | 1.74 |
| 13.89 | −60.86 | −51.02 | −63.98 | −58.62 | 6.76 |
| 4.63 | −74.57 | −70.83 | −81.17 | −75.52 | 5.24 |
| 1.54 | −63.98 | −69.71 | −46.41 | −60.03 | 12.14 |
| 0.51 | −71.45 | −77.56 | −73.70 | −74.24 | 3.09 |
| 0.17 | −49.03 | −43.54 | −39.93 | −44.17 | 4.58 |
| 0.06 | −39.68 | −40.18 | −37.32 | −39.06 | 1.53 |

TABLE 20
72 hours-Enantiomer A

| | Cell | Cell | Cell | | |
|---|---|---|---|---|---|
| Concentration | death | death #2 | death | | |
| (μM) | #1 (%) | (%) | #3 (%) | MEAN | SD |
| 125.00 | 75.56 | 74.94 | 74.82 | 75.11 | 0.40 |
| 41.67 | 9.78 | 23.24 | 14.02 | 15.68 | 6.88 |
| 13.89 | −45.54 | −47.91 | −30.21 | −41.22 | 9.60 |
| 4.63 | −76.31 | −66.59 | −54.63 | −65.85 | 10.86 |
| 1.54 | −46.91 | −52.39 | −53.51 | −50.94 | 3.53 |
| 0.51 | −63.73 | −69.58 | −63.23 | −65.51 | 3.53 |
| 0.17 | −43.42 | −40.55 | −38.31 | −40.76 | 2.56 |
| 0.06 | −39.43 | −40.43 | −33.95 | −37.94 | 3.49 |

TABLE 21
72 hours-Enantiomer B

| | Cell | Cell | Cell | | |
|---|---|---|---|---|---|
| Concentration | death | death #2 | death | | |
| (μM) | #1 (%) | (%) | #3 (%) | MEAN | SD |
| 125.00 | 64.88 | 61.78 | 57.24 | 61.30 | 3.85 |
| 41.67 | 11.03 | 11.96 | 14.65 | 12.55 | 1.88 |
| 13.89 | −33.31 | −22.56 | −34.75 | −30.21 | 6.66 |
| 4.63 | −45.81 | −40.03 | −52.74 | −46.19 | 6.37 |
| 1.54 | −52.64 | −40.34 | −51.19 | −48.05 | 6.72 |
| 0.51 | −47.57 | −52.84 | −52.74 | −51.05 | 3.01 |
| 0.17 | −33.20 | −39.41 | −29.79 | −34.13 | 4.87 |
| 0.06 | −22.56 | −30.41 | −23.49 | −25.49 | 4.29 |

Morphological analysis through optical microscopy did not show any deleterious effect at any concentration. No compound precipitation was observed at any concentration.

These results indicate that at higher concentrations, dibenzyl butyl lactone derivative enantiomers A and B exhibit increased prostate cancer cell death as compared to enterolactone at both 24 hours and 72 hours post-treatment.

### Example 5

### The Dibenzyl Butyl Lactone Derivative Compound Inhibits Telomerase in Prostate Cancer Cells

Telomeric repeat amplification protocol (TRAP) was modified for quantitative real-time PCR (Q-TRAP) and was used to determine the effect of dibenzyl butyl lactone derivative on telomerase activity in prostate cancer cell. The same methods as used in Example 3 were employed. The same abbreviation scheme as presented in Table 7 is used throughout this example as well.

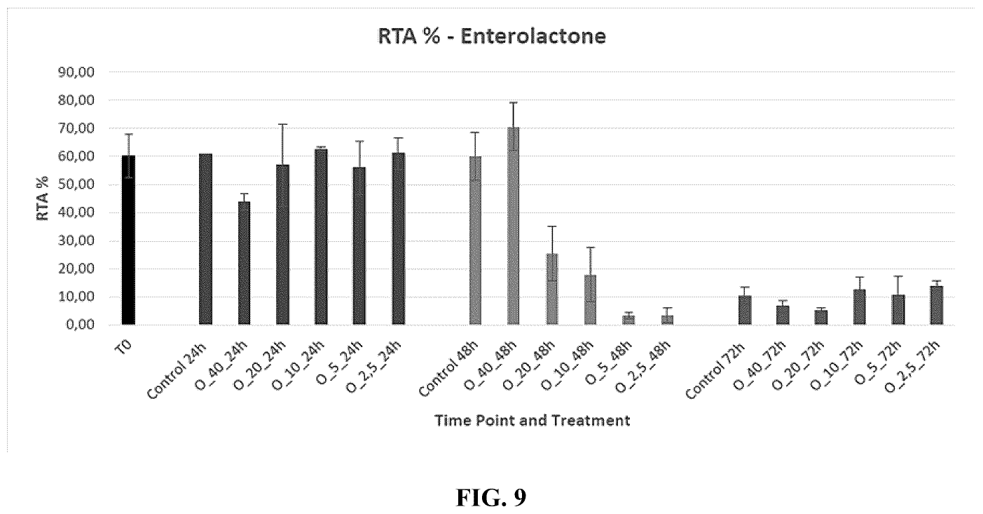

Calculated RTA values for all enterolactone treated samples are shown in Table 22. Q-TRAP results are shown in FIG. 9.

TABLE 22
Telomerase activity (RTA %) Enterolactone

| | 0 Hours | | 24 Hours | | 48 Hours | | 72 Hours | |
|---|---|---|---|---|---|---|---|---|
| | RTA | | RTA | | RTA | | RTA | |
| | (%) | SD | (%) | SD | (%) | SD | (%) | SD |
| Control | 60.14 | 7.76 | 60.64 | 0.21 | 59.88 | 8.53 | 10.32 | 3.11 |
| O_40 | | | 43.75 | 2.88 | 70.42 | 8.54 | 6.57 | 2.04 |
| O_20 | | | 56.77 | 14.63 | 25.39 | 9.70 | 5.15 | 0.81 |
| O_10 | | | 62.45 | 0.76 | 17.89 | 9.69 | 12.63 | 4.23 |
| O_5 | | | 55.90 | 9.52 | 3.31 | 1.17 | 10.65 | 6.64 |
| O_2.5 | | | 61.02 | 5.60 | 3.50 | 2.56 | 13.88 | 1.73 |

Data were grouped by time point of treatment and statistical analysis for grouped data was applied using t-test comparing with a control condition. Results are shown in Table 23.

TABLE 23
T-Student analysis indicates if there are significant differences among the Q-TRAP
results (RTA) compared to control condition. Significant differences are indicated in the
“Significance” column. From lowest to highest significance: No: non-significant; Yes (*):
p < 0.05; Yes (**): p < 0.01; Yes (***): p < 0.001; Yes (****): p < 0.0001.
T-Student Analysis-Relative Telomerase Activity
Enterolactone

| 24 hours | RTA (p Value) | Significance |
|---|---|---|
| Control vs. O_40 | 0.0143 | Yes (*) |
| Control vs. O_20 | 0.7441 | No |
| Control vs. O_10 | 0.0839 | No |
| Control vs. O_5 | 0.5544 | No |
| Control vs. O_2.5 | 0.9329 | No |
| 48 hours | RTA (p Value) | Significance |
| Control vs. O_40 | 0.2688 | No |
| Control vs. O_20 | 0.0098 | Yes (**) |
| Control vs. O_10 | 0.0049 | Yes (**) |
| Control vs. O_5 | 0.0003 | Yes (***) |
| Control vs. O_2.5 | 0.0004 | Yes (***) |
| 72 hours | RTA (p Value) | Significance |
| Control vs. O_40 | 0.2381 | No |
| Control vs. O_20 | 0.1156 | No |
| Control vs. O_10 | 0.5239 | No |
| Control vs. O_5 | 0.9419 | No |
| Control vs. O_2.5 | 0.1580 | No |

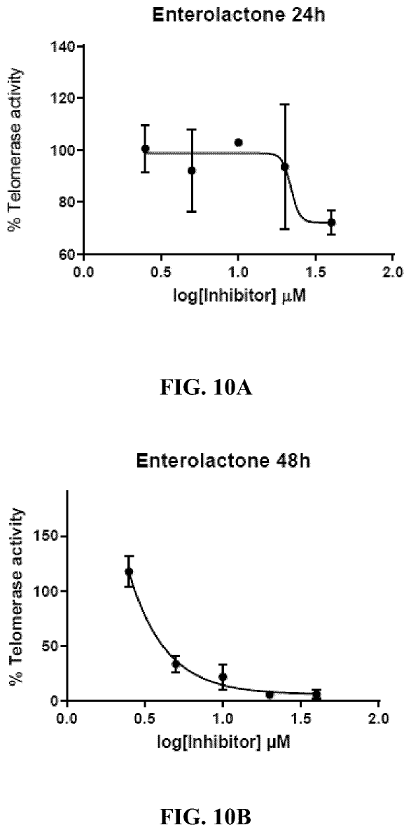

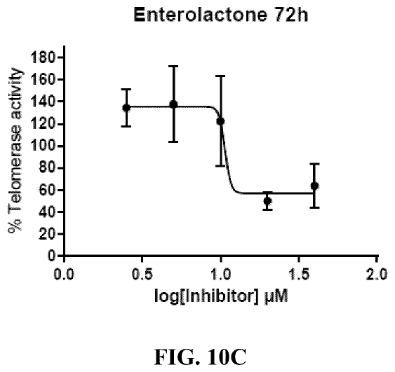

Graphs of IC50 of enterolactone at 24, 48, and 72 hours were generated using a nonlinear regression and plotted as log [inhibitor] vs. response (FIGS. 10A-C). The average of telomerase activity from replicates were calculated for each concentration of enterolactone at 24, 48 and 72h and compared to the average value of the no-drug control to generate the percent (%) of telomerase activation and plotted against the treatment concentration to determine the drug concentrations for each formulation to achieve 50% inhibition (IC50 value), as shown in Table 24.

TABLE 24
IC50 μM-Enterolactone

| | 24 h | 48 h | 72 h |
|---|---|---|---|
| Enterolactone | 21.92 | 0.05 | 10.71 |

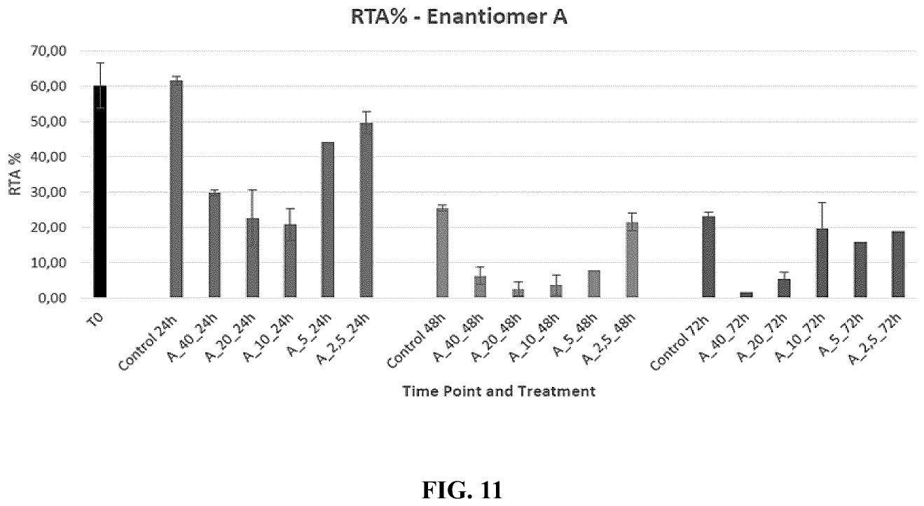

Calculated RTA values for all dibenzyl butyl lactone derivative enantiomer A treated samples are shown in Table 25. Q-TRAP results are shown in FIG. 11.

TABLE 25
Telomerase Activity (RTA %) - Enantiomer A

| | 0 Hours | | 24 Hours | | 48 Hours | | 72 Hours | |
|---|---|---|---|---|---|---|---|---|
| | RTA | | RTA | | RTA | | RTA | |
| | (%) | SD | (%) | SD | (%) | SD | (%) | SD |
| Control | 60.14 | 7.76 | 61.53 | 6.32 | 25.39 | 3.05 | 23.04 | 2.45 |
| A_40 | | | 29.78 | 8.38 | 6.23 | 2.75 | 1.48 | 0.31 |
| A_20 | | | 22.61 | 1.29 | 2.51 | 0.98 | 5.20 | 1.36 |
| A_10 | | | 20.76 | 0.75 | 3.60 | 2.47 | 19.40 | 0.00 |
| A_5 | | | 44.11 | 7.88 | 7.86 | 1.91 | 15.77 | 2.12 |
| A_2.5 | | | 49.67 | 4.59 | 21.48 | 2.87 | 18.63 | 7.61 |

Data were grouped by time point of treatment and statistical analysis for grouped data was applied using t-test comparing with a control condition. Results are shown in Table 26.

TABLE 26
T-Student analysis indicates if there are significant differences among the Q-TRAP
results (RTA) compared to control condition. Significant differences are indicated in the
“Significance” column. From lowest to highest significance: No: non-significant; Yes (*):
p < 0.05; Yes (**): p < 0.01; Yes (***): p < 0.001; Yes (****): p < 0.0001.
T-Student Analysis-Relative Telomerase Activity
Enantiomer A

| 24 hours | RTA (p Value) | Significance |
|---|---|---|
| Control vs. A_40 | 0.0063 | Yes (**) |
| Control vs. A_20 | 0.0005 | Yes (***) |
| Control vs. A_10 | 0.0033 | Yes (**) |
| Control vs. A_5 | 0.0405 | Yes (*) |
| Control vs. A_2.5 | 0.1112 | No |
| 48 hours | RTA (p Value) | Significance |
| Control vs. A_40 | 0.0222 | Yes (*) |
| Control vs. A_20 | 0.0010 | Yes (**) |
| Control vs. A_10 | 0.0030 | Yes (**) |
| Control vs. A_5 | 0.0204 | Yes (*) |
| Control vs. A_2.5 | 0.3178 | No |
| 72 hours | RTA (p Value) | Significance |
| Control vs. A_40 | 0.0065 | Yes (**) |
| Control vs. A_20 | 0.0122 | Yes (*) |
| Control vs. A_10 | 0.1708 | No |
| Control vs. A_5 | 0.0379 | Yes (*) |
| Control vs. A_2.5 | 0.5173 | No |

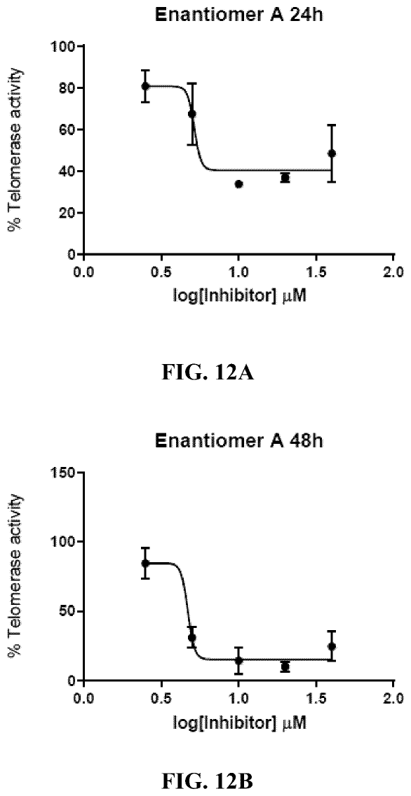

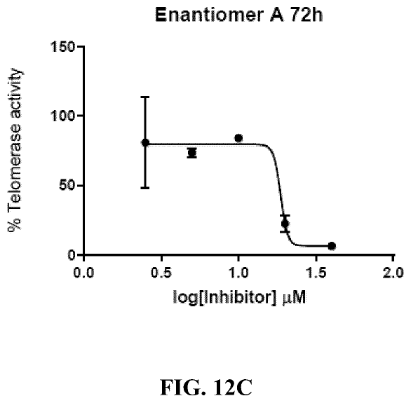

Graphs of IC50 of dibenzyl butyl lactone derivative enantiomer A at 24, 48, and 72 hours were generated using a nonlinear regression and plotted as log [inhibitor] vs. response (FIGS. 12A-C). The average of telomerase activity from replicates were calculated for each concentration of dibenzyl butyl lactone derivative enantiomer A at 24, 48 and 72h and compared to the average value of the no-drug control to generate the percent (%) of telomerase activation and plotted against the treatment concentration to determine the drug concentrations for each formulation to achieve 50% inhibition (IC50 value), as shown in Table 27.

TABLE 27
IC50 μM-Enantiomer A

| | 24 h | 48 h | 72 h |
|---|---|---|---|
| Enantiomer A | 5.18 | 4.71 | 18.73 |

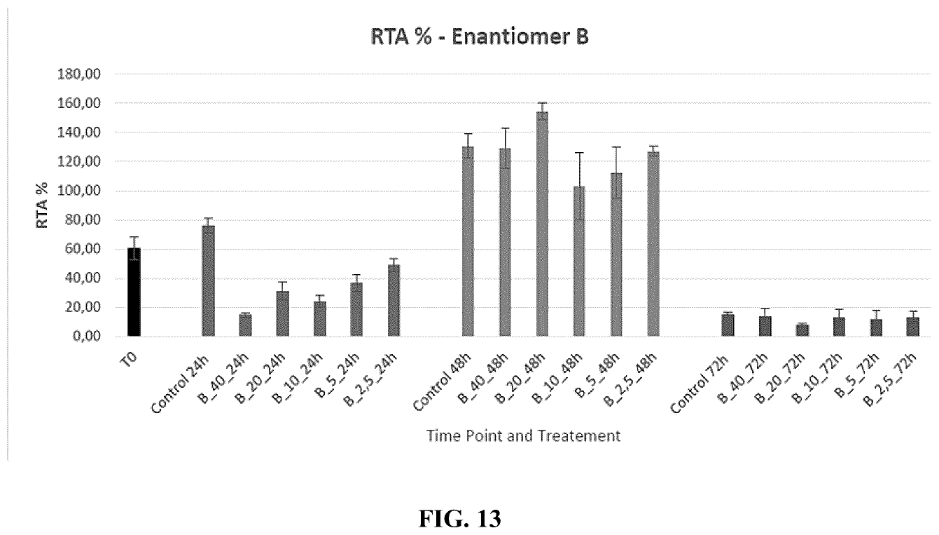

Calculated RTA values for all dibenzyl butyl lactone derivative enantiomer B treated samples are shown in Table 28. Q-TRAP results are shown in FIG. 13.

TABLE 28
Telomerase activity (RTA %) - Enantiomer B

| | 0 Hours | | 24 Hours | | 48 Hours | | 72 Hours | |
|---|---|---|---|---|---|---|---|---|
| | RTA | | RTA | | RTA | | RTA | |
| | (%) | SD | (%) | SD | (%) | SD | (%) | SD |
| Control | 60.14 | 7.76 | 75.92 | 5.23 | 130.28 | 8.44 | 14.62 | 1.85 |
| B_40 | | | 14.44 | 1.35 | 128.83 | 13.91 | 13.49 | 5.47 |
| B_20 | | | 31.05 | 6.16 | 154.17 | 5.77 | 7.53 | 1.18 |
| B_10 | | | 23.87 | 4.18 | 102.96 | 22.92 | 12.58 | 5.96 |
| B_5 | | | 36.48 | 5.61 | 112.17 | 17.77 | 11.67 | 6.14 |
| B_2.5 | | | 48.73 | 4.20 | 126.77 | 3.54 | 13.05 | 4.29 |

Data were grouped by time point of treatment and statistical analysis for grouped data was applied using t-test comparing with a control condition. Results are shown in Table 29.

TABLE 29
T-Student analysis indicates if there are significant differences among the Q-TRAP
results (RTA) compared to control condition. Significant differences are indicated in the
“Significance” column. From lowest to highest significance: No: non-significant; Yes (*):
p < 0.05; Yes (**): p < 0.01; Yes (***): p < 0.001; Yes (****): p < 0.0001.
T-Student Analysis-Relative Telomerase Activity
Enantiomer B

| 24 hours | RTA (p Value) | Significance |
|---|---|---|
| Control vs. B_40 | 0.0002 | Yes (***) |
| Control vs B_20 | 0.0036 | Yes (**) |
| Control vs. B_10 | 0.0011 | Yes (**) |
| Control vs. B_5 | 0.0184 | Yes (**) |
| Control vs. B_2.5 | 0.0291 | Yes (*) |
| 48 hours | RTA (p Value) | Significance |
| Control vs. B_40 | 0.9058 | No |
| Control vs B_20 | 0.0807 | No |
| Control vs. B_10 | 0.2545 | No |
| Control vs. B_5 | 0.2854 | No |
| Control vs. B_2.5 | 0.6412 | No |
| 72 hours | RTA (p Value) | Significance |
| Control vs. B_40 | 0.8074 | No |
| Control vs B_20 | 0.0446 | Yes (*) |
| Control vs. B_10 | 0.6847 | No |
| Control vs. B_5 | 0.5731 | No |
| Control vs. B_2.5 | 0.6818 | No |

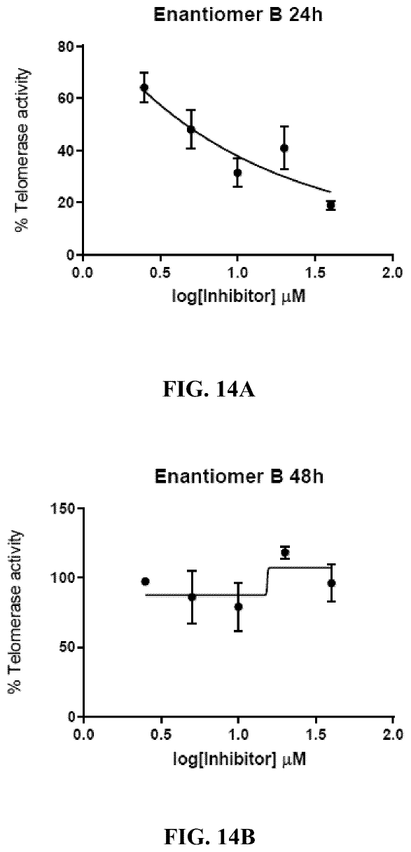

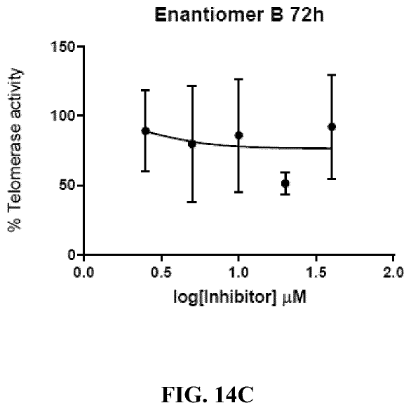

Graphs of IC50 of dibenzyl butyl lactone derivative enantiomer B at 24, 48, and 72 hours were generated using a nonlinear regression and plotted as log [inhibitor] vs. response (FIGS. 14A-C). The average of telomerase activity from replicates were calculated for each concentration of dibenzyl butyl lactone derivative enantiomer B at 24, 48 and 72 h and compared to the average value of the no-drug control to generate the percent (%) of telomerase activation and plotted against the treatment concentration to determine the drug concentrations for each formulation to achieve 50% inhibition (IC50 value), as shown in Table 30.

TABLE 30
IC50 μM-Enantiomer B

| | 24 h | 48 h | 72 h |
|---|---|---|---|
| Enantiomer | 2.987 | 15.38 | 2.384 |
| B | | | |

### Discussion

At 24 hours all groups treated with enterolactone showed lower telomerase activity levels compared to control however only the highest concentration presented a significant difference. At 48 hours four treated groups presented a reduction in RTA compared to the control untreated group. All of these changes were found to be significant. At 72 hours no significant differences were detected between control and treated groups.

At 24 hours, all groups treated with dibenzyl butyl lactone derivative enantiomer A showed lower telomerase activity levels compared to control. The differences were significant for all groups except for the lowest concentration. At 48 hours, all treated groups showed lower telomerase activity levels compared to control. The differences were significant for all groups except for the lowest concentration. At 72 hours, all treated groups showed lower telomerase activity levels compared to control. The differences were significant for the two highest concentrations.

At 24 hours, all groups treated with dibenzyl butyl lactone derivative enantiomer B showed lower telomerase activity levels compared to control with all of the differences being significant. At 48 hours, the groups treated with concentrations 40, 10, 5 and 2.5 showed lower telomerase activity levels compared to control. However, the differences were not found to be significant. At 72 hours, the telomerase activity dropped to basal levels without any particular differences between the treated and control groups.

As a whole, these results show that dibenzyl butyl lactone derivative enantiomer A exhibits a telomerase inhibition effect in a PC3 prostate cancer cell line that is more prolonged throughout all time points. Enantiomer B exhibits a telomerase inhibition effect at 24 hours.

### Example 6

The Dibenzyl Butyl Lactone Derivative Compound Exhibits Low Developmental Toxicity Potential (dTP)

The dibenzyl butyl lactone derivative enantiomer A and B were compared to enterolactone and tested to determine toxicity using a human pluripotent stem (hPS) cell-based assay that predicts the developmental toxicity potential. The assay uses the metabolic perturbation of two biomarkers, omnithine and cystine, in a ratio (o/c ratio) to predict the concentration at which a test article shows developmental toxicity potential (dTP). The current study was conducted using human induced pluripotent stem (iPS) cells.

The impact of test article exposure on ornithine and cystine metabolism in human induced pluripotent stem (iPS) cells was measured for three test articles (dibenzyl butyl lactone derivative enantiomer A, dibenzyl butyl lactone derivative enantiomer B, and enterolactone) and prediction of the potential for developmental toxicity was made through application of the hPS cell-based assay. Exposure spanned a range of eight treatment levels per test article ranging from 0.03-100 μM.

The dTP (o/c ratio) and toxicity potential (TP, iPS cell viability) effect concentrations are summarized in Table 31.

TABLE 31
Toxicity Results

| | dTP Concentration | TP Concentration |
|---|---|---|
| Test Article | (μM) | (μM) |
| Enantiomer B | 15.1 | 49.8 |
| Enantiomer A | 21.4 | 34.8 |
| (+/−) Enterolactone | 21.8 | 230.8 |

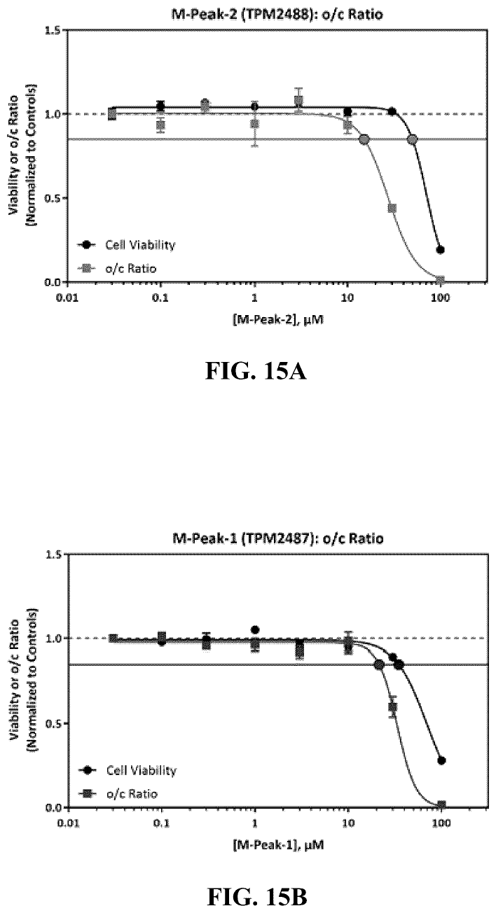

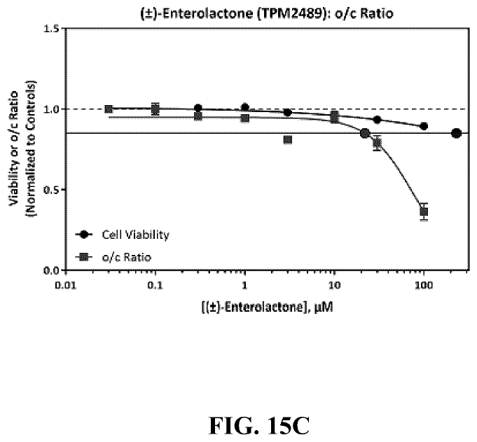

The observed decrease in the o/c ratio following exposure to each test article is indicative of the potential for developmental toxicity and/or embryo lethality in vivo at or above the dTP concentration. FIGS. 15A-C show the o/c ratio of dibenzyl butyl lactone derivative enantiomer A (FIG. 15A), dibenzyl butyl lactone derivative enantiomer B (FIG. 15B), and (+/−) enterolactone (FIG. 15C). The graphs in FIGS. 15A-C show the cell viability and o/c/ ratio over concentrations of 0.03-100 μM with the straight horizontal line illustrating the developmental toxicity threshold of 0.85. Dibenzyl butyl lactone derivative enantiomer B and (+/−) enterolactone decreased the o/c ratio independent of changes in cell viability (FIGS. 15A and 15C). Dibenzyl butyl lactone derivative enantiomer A decreased the o/c ratio at concentrations similar to those that impacted cell viability. dTP and TP are within 3-fold; (FIG. 15B).

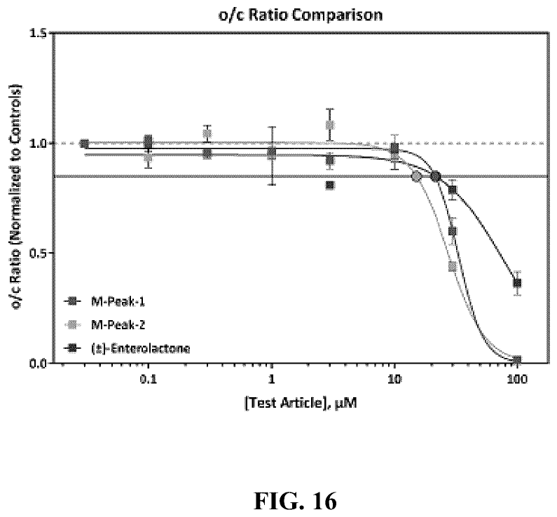

All three test articles, dibenzyl butyl lactone derivative enantiomers A and B and (+/−) enterolactone, elicited a change in the o/c ratio at similar concentrations. In fact, as shown in FIG. 16, the dTP concentrations are similar (within 2-fold) between all three compounds. However, (+/−) enterolactone was less cytotoxic than dibenzyl butyl lactone derivative enantiomers A and B. Yet, dibenzyl butyl lactone derivative enantiomers A and B TP concentrations, as shown in Table 31, are 4-6-fold lower than (+/−) enterolactone TP.

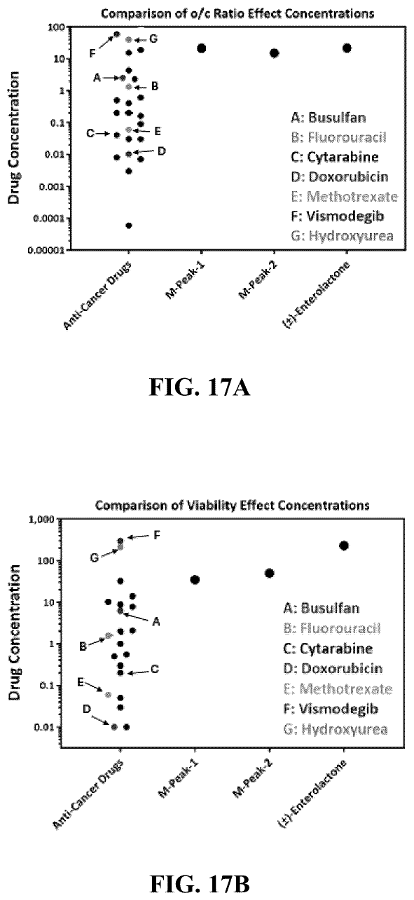

The dibenzyl butyl lactone derivative enantiomers A and B and (+/−) enterolactone were also compared to 7 compounds that relate to 24 marketed anti-cancer drugs which were all evaluated in the (hPS) cell-based assay. The dTP and TP concentrations for the dibenzyl butyl lactone derivative enantiomers A and B and (+/−) enterolactone were evaluated and compared to the dTP and TP concentrations for the marketed anti-cancer drugs busulfan, fluorouracil, cytarabine, doxorubicin, methotrexate, vismodegib, and hydroxyurea. FIGS. 17A and 17B demonstrate the dTP (FIG. 17A) and TP (FIG. 17B) concentrations observed.

From these graphs, it is demonstrated that dibenzyl butyl lactone derivative enantiomers A and B were higher than >90% of the anti-cancer drugs that have been tested in this assay, which can indicate a lower potency, or a lower potential for developmental toxicity. It is important to note that all currently marketed anti-cancer drugs have shown potential for developmental toxicity in vivo and are expected to cause harm to the developing fetus based on their mechanisms of action. Yet, the results indicate that dibenzyl butyl lactone derivative enantiomers A and B show a lesser potential for toxicity than many anti-cancer drugs, especially busulfan, fluorouracil, cytarabine, doxorubicin, and methotrexate, and show similar dTP and TP concentrations as compared to (+/−) enterolactone. Thus, these results indicate that the dibenzyl butyl lactone derivative enantiomers A and B exhibit similar toxicity as enterolactone and thus has a decreased likelihood in toxicity as compared to many current cancer drugs on the market.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the disclosure in diverse forms thereof.

What is claimed is:

1. A pharmaceutical compound represented by the structure of formula I: 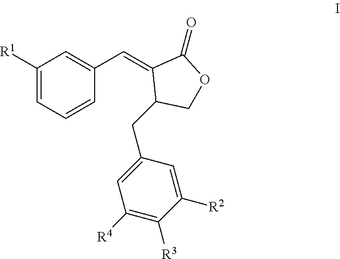
wherein:
R¹ and R² are independently a hydrogen, hydroxyl group or C1-22 alkyl group; and
R³ and R⁴ are an ethylene oxide, dioxirane, oxetane, dioxetane, dioxolane, dihydrofuran, or furan,
or its enantiomer or racemic mixture thereof, or a pharmaceutically acceptable salt thereof.

2. The compound of claim 1, wherein R¹ is a hydroxyl group or C1-22 alkyl group, R² is a hydrogen, and R³ and R⁴ are an ethylene oxide, dioxirane, oxetane, dioxetane, dioxolane, dihydrofuran, or furan.

3. The compound of claim 1, wherein R¹ is a hydroxyl group, R² is a hydrogen, and R³ and R⁴ are an ethylene oxide, dioxirane, oxetane, dioxetane, dioxolane, dihydrofuran, or furan.

4. The compound of claim 1, wherein R¹ is a hydroxyl group, R² is a hydrogen, and R³ and R⁴ form a dioxolane.

5. A pharmaceutical composition comprising:
a compound represented by the structure of formula I: 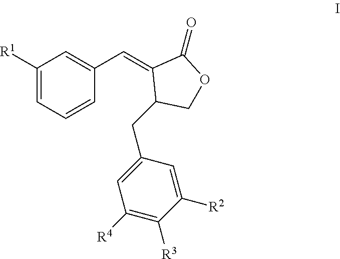
wherein:
R¹ and R² are independently a hydrogen, hydroxyl group or C1-22 alkyl group; and
R³ and R⁴ are an ethylene oxide, dioxirane, oxetane, dioxetane, dioxolane, dihydrofuran, or furan,
or its enantiomer or racemic mixture thereof, or a pharmaceutically acceptable salt thereof; and
at least one pharmaceutically acceptable excipient.

6. The composition of claim 5, wherein R¹ is a hydroxyl group or C1-22 alkyl group, R² is a hydrogen, and R³ and R⁴ are an ethylene oxide, dioxirane, oxetane, dioxetane, dioxolane, dihydrofuran, or furan.

7. The composition of claim 5, wherein R¹ is a hydroxyl group, R² is a hydrogen, and R³ and R⁴ are an ethylene oxide, dioxirane, oxetane, dioxetane, dioxolane, dihydrofuran, or furan.

8. The composition of claim 5, wherein R¹ is a hydroxyl group, R² is a hydrogen, and R³ and R⁴ form a dioxolane.

9. The composition of claim 5, wherein the pharmaceutically acceptable excipient comprises a carrier, adjuvant, diluent, buffer, stabilizer, preservative, and/or lubricant.

10. The composition of claim 5, wherein the composition is formulated for oral, intravenous, or intramuscular use.

11. A method of treating cancer in a subject, comprising administering to the subject a composition comprising a compound represented by the structure of formula I: 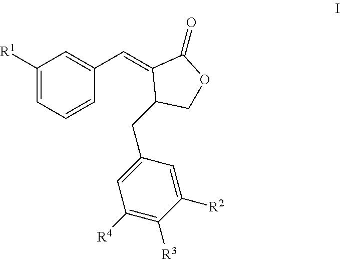
wherein:
R¹ and R² are independently a hydrogen, hydroxyl group or C1-22 alkyl group; and
R³ and R⁴ are an ethylene oxide, dioxirane, oxetane, dioxetane, dioxolane, dihydrofuran, or furan,
or its enantiomer or racemic mixture thereof, or a pharmaceutically acceptable salt thereof.

12. The method of claim 11, wherein R¹ is a hydroxyl group or C1-22 alkyl group, R² is a hydrogen, and R³ and R⁴ are an ethylene oxide, dioxirane, oxetane, dioxetane, dioxolane, dihydrofuran, or furan.

13. The method of claim 11, wherein R¹ is a hydroxyl group, R² is a hydrogen, and R³ and R⁴ form a dioxolane.

14. The method of claim 11, wherein the cancer is breast cancer or prostate cancer.

15. The method of claim 11, wherein the cancer is metastatic cancer.

16. The method of claim 11, wherein the composition is administered orally, intravenously, or intramuscularly.

17. The method of claim 11, wherein the composition is administered in combination with a chemotherapeutic agent.

18. The method of claim 11, wherein the composition further comprises a pharmaceutically acceptable excipient.

19. The method of claim 18, wherein the pharmaceutically acceptable excipient comprises a carrier, adjuvant, diluent, buffer, stabilizer, preservative, and/or lubricant.

20. The method of claim 11, wherein the subject is human.
